(19) United States
(12) Patent Application Publication
(10) Pub. No.: US 2008/0286314 A1
(43) Pub. Date: Nov. 20, 2008

(54) Dendritic cells loaded with heat shocked melanoma cell bodies

(75) Inventors: Anna Karolina Palucka, Dallas, TX (US); Jacques Banchereau, Dallas, TX (US)

Correspondence Address: CHALKER FLORES, LLP, 2711 LBJ FRWY, Suite 1036, DALLAS, TX, 75234, US

(73) Assignee: Baylor Research Institute, Dallas, TX (US)

(21) Appl. No.: 11/932,095

(22) Filed: Oct. 31, 2007

Related U.S. Application Data
(62) Division of application No. 11/257,951, filed on Oct. 25, 2005.
(60) Provisional application No. 60/621,957, filed on Oct. 25, 2004.

(51) Int. Cl. A61K 39/00; C12N 5/02; A61P 35/04

(52) U.S. Cl. 424/277.1; 435/375

(57) ABSTRACT

The present invention includes compositions and methods for the isolation, purification and preparation of immunogenic antigens for the production of customized cancer vaccines that include dendritic cells that are contacted with an antigen that includes heat-shocked cancer cells.

This application claims priority to U.S. Provisional Patent Application Ser. No. 60/621,957, filed Oct. 25, 2004, the entire contents of which are incorporated herein by reference. Without limiting the scope of the invention, its background is described in connection with vaccination.

### TECHNICAL FIELD OF THE INVENTION

This invention relates to compositions and methods for inducing immunity to cancer, and more particularly, to the preparation, treatment and methods of making immunogenic cancer-specific antigens.

### BACKGROUND OF THE INVENTION

The activation of the adaptive immune response against a specific target remains one of the most complex and sought-after goals in immunology. A key cell in the immune activation process is the dendritic cell, due to its ability to efficiently process and present antigens on both Major Histocompatibility Complex (MHC) class I and II molecules. A number of factors, genetic and environmental, affect the ability of the immune response to recognize and respond to processed antigens presented by antigen presenting cells (APCs) such as dendritic cells.

In the case of a cytotoxic immune response, a classic class I pathway model is the use of influenza virus for inducing CD8+ cytotoxic T lymphocytes (CTLs), which requires the accurate processing, transit and delivery of peptides with the proper antigenic epitope to the cell surface. Upon recognition by sufficient T cell receptors (TcRs) and the proper co-stimulation, an antigen-specific immune response is possible. Although dendritic cells efficiently activate class I-restricted CTLs, access to the MHC class I pathway for induction of CD8+ T cells normally requires synthesis of antigen. Many models include the use of antigens and antigen delivery systems that efficiently deliver antigen to the MHC class I-restricted antigen presentation pathway to generate antigen-specific CD8+ T cell responses.

Other approaches for antigen presentation include the delivery of exogenous antigen to the MHC I processing pathway of dendritic cells, e.g., by coupling antigens to potent adjuvants, osmotic lysis endocytosed antigen and insertion of antigen in pH-sensitive liposomes. While useful for in vitro analysis, these approaches have found difficulty in therapeutic applications. To date, dendritic cells may be pulsed directly with exogenous antigens using whole cells in viable or irradiated forms, membrane preparations, apoptotic cells or cell bodies and antigens purified from natural sources or expressed as recombinant products, see e.g., WO 94/02156 and U.S. Pat. No. 6,602,709. These prior methods, however, do not recognize forms of cell death or the processing pathways antigens from dead or dying cells access in the dendritic cell system.

One example of the use of dendritic cells is taught in U.S. Pat. No. 6,936,468, issued to Robbins, et al., for the use of tolerogenic dendritic cells for enhancing tolerogenicity in a host and methods for making the same. Briefly, tolerogenic mammalian dendritic cells (DCs) and methods for the production of the tolerogenic DCs are disclosed. A method for enhancing tolerogenicity in a host is provided by administering the tolerogenic mammalian DCs to a host. The tolerogenic DCs include an oligodeoxyribonucleotide (ODN) that has one or more NF-κB binding sites. Tolerogenic DCs may also include a viral vector, e.g., an adenoviral vector, which does not affect the tolerogenicity of the tolerogenic DCs when present therein. Enhanced tolerogenicity in a host is said to be useful for prolonging foreign graft survival and for treating inflammatory related diseases, such as autoimmune diseases.

Yet another use of dendritic cells is taught in U.S. Pat. No. 6,734,014, issued to Hwu, et al., for methods and compositions for transforming dendritic cells and activating T cells. Briefly, recombinant dendritic cells are made by transforming a stem cell and differentiating the stem cell into a dendritic cell. The resulting dendritic cell is said to be an antigen presenting cell which activates T cells against MHC class I-antigen targets. The disclosure also includes kits, assays and therapeutics based on the activation of T cells by the recombinant dendritic cell. Cancer, viral infections and parasitic infections are said to be ameliorated by the recombinant dendritic cells, or corresponding activated T cells.

Antigens for use in dendritic cell loading are taught in, e.g., U.S. Pat. No. 6,602,709, issued to Albert, et al. This patent teaches methods for use of apoptotic cells to deliver antigen to dendritic cells for induction or tolerization of T cells. The methods and compositions are said to be useful for delivering antigens to dendritic cells that are useful for inducing antigen-specific cytotoxic T lymphocytes and T helper cells. The disclosure includes assays for evaluating the activity of cytotoxic T lymphocytes. The antigens targeted to dendritic cells are apoptotic cells that may also be modified to express non-native antigens for presentation to the dendritic cells. The dendritic cells are said to be primed by the apoptotic cells (and fragments thereof) capable of processing and presenting the processed antigen and inducing cytotoxic T lymphocyte activity or may also be used in vaccine therapies.

Finally, U.S. Pat. No. 6,455,299, issued to Steinman, et al., teaches methods of use for viral vectors to deliver antigen to dendritic cells. Methods and compositions are said to be useful for delivering antigens to dendritic cells, which are then useful for inducing T antigen specific cytotoxic T lymphocytes. The disclosure provides assays for evaluating the activity of cytotoxic T lymphocytes. Antigens are provided to dendritic cells using a viral vector such as influenza virus that may be modified to express non-native antigens for presentation to the dendritic cells. The dendritic cells are infected with the vector and are said to be capable of presenting the antigen and inducing cytotoxic T lymphocyte activity or may also be used as vaccines.

### SUMMARY OF THE INVENTION

It has now been found that monocyte-derived DCs loaded with either heat shocked killed tumor cells or with killed tumor bodies overexpressing heat shock proteins or peptides can be used to prime naïve T cells and induce their differentiation to more powerful, highly efficient antigen-specific cytotoxic T lymphocytes (CTLs). Compositions, methods of use and methods for preparation of these DCs are disclosed herein.

The present invention includes compositions and methods for inducing immunity to cancer in a patient by using isolated and purified antigen presenting cells primed by exposure to one or more heat-shocked and killed cancer cells. The antigen presenting cells may be professional antigen presenting cells, e.g., dendritic cells. Generally, the antigen presenting cells are loaded with heat-shocked, heat-killed cancer cells, e.g., cancer cells are isolated from a patient and/or allogeneic cancer cells or cell lines. The heat-shocked and killed cancer cells are internalized and processed by the antigen presenting cells for at least 2 hours.

The present invention also includes methods for inducing immunity to cancer in a patient by heat-shocking one or more cancer cells at a temperature of at least about 42° C. for at least two hours to form heat shocked cancer cells; killing the heat shocked cancer cells to form heat shocked, killed cancer cells; incubating one or more antigen presenting cells isolated from the patient with the heat shocked, killed cancer cells for at least three hours; and administering one or more isolated, loaded antigen presenting cells to the patient. The antigen presenting cells maybe matured with one or more cytokines prior to administering to the patient.

Another method of the present invention includes inducing immunity to cancer in a patient by obtaining antigen presenting cells from the patient; incubating allogeneic cancer cells at a temperature of at least 42° C. for at least two hours to form heat shocked allogeneic cancer cells; killing the heat shocked allogeneic cancer cells to form heat shocked, killed allogeneic cancer cells; exposing the antigen presenting cells to the heat shocked, killed allogeneic cancer cells for at least three hours to form loaded antigen presenting cells; maturing the isolated, loaded antigen presenting cells; and administering the isolated, loaded antigen presenting cells to the patient. The skilled artisan will recognize that the antigen presenting cells may be dendritic cells in various stages of maturation and the heat shocked, killed cancer cells may be internalized by the antigen presenting cells (e.g., the dendritic cells) as the antigen presenting cells are matured with one or more cytokines. Examples of allogeneic cancer cells may be selected from Table II.

Yet another method of preparing immunogenic isolated antigen presenting cells may include the steps of isolating antigen presenting cells from a subject; preparing an antigen by stressing one or more cancer cells and killing the cancer cells; loading the antigen presenting cells with the antigen for at least three hours; and isolating and purifying the loaded antigen presenting cells. The cancer cells may be stressed by a method selected from the group consisting of heat shock, cold shock, glucose deprivation, oxygen deprivation, exposure to at least one drug that alter cell metabolism, and exposure to at least one cytotoxic drug prior to killing the cancer cells. The cancer cells may be autologous or allogeneic cancer cells. In fact, the step of loading the antigen presenting cells with the antigen may also be conducted under heat shock conditions. In one simple step, the present invention includes a method of increasing the expression of tumor antigens in stressed and killed cancer cells by stressing the cancer cells prior to killing the cancer cells. The cancer cells may be stressed by heat shock, cold shock, glucose deprivation, oxygen deprivation, exposure to at least one drug that alter cell metabolism, and/or exposure to at least one cytotoxic drug prior to killing the cancer cells.

Yet another embodiment of the present invention includes a method of increasing the antigenicity of tumor antigens in antigen presenting cells loaded with stressed and killed cancer cells by stressing the cancer cells and killing the cancer cells prior to exposure of antigen presenting cells to the stressed and killed cancer cells. The antigenicity of the cancer cells may be increased by stressing them by heat shock, cold shock, glucose deprivation, oxygen deprivation, exposure to at least one drug that alters cell metabolism, and exposure to at least one cytotoxic drug prior to killing the cancer cells. As such, the present invention includes an antigen that includes heat shocked cancer cells and portions thereof.

The antigen of the present invention may be prepared by a method that includes heat-treating one or more cancer cell lines and killing the cells with a cell death inducing agent. The cell death may be accomplished by killing agents comprises betulinic acid, paclitaxel, camptothecin, ellipticine, mithramycin A, etoposide, vinblastine, vincristine, ionomycin and combinations thereof. Alternatively or in conjunction, cell death may be achieved by exposing the cancer cells to radiation, heat, cold, osmotic shock, pressure, grinding, shearing, ultrasound, drying, freeze spraying, puncturing, starving and combinations thereof. The cancer cell may heat treated for 2, 4, 6 or 8 hours and after killing may be stored in lyophilized, heat-dried, vacuum dried, heat-vacuum dried, frozen by evaporative precipitation into aqueous solution (EPAS), spray freezing into liquid (SFL), antisolvent precipitation or freeze sprayed form prior to use. When used as part of a kit, the antigen may further include a contained with a diluent for resuspending the antigen, e.g., saline, pH buffered saline, saline with one or more cytokines, adjuvants or antigens and/or any other solution for resuspension.

In one embodiment, the cancer cell is defined further as being a hot melanoma and portions thereof. The antigen may include heat-shocked and killed cancer cells and portions thereof, e.g., with one or more antigen presenting cells and/or an adjuvant. The cancer cells may also be heat-killed, or killed by any of a variety of known methods. One method is the direct killing of the cell by chemical, mechanical and irradiative methods. Yet another embodiment includes the use or programmed cell death or apoptosis, which may also be used with the present invention after heat-shock of the cells to increase the antigenicity of the cancer cells. The heat shocked cancer cells and portions thereof may be killed by betulinic acid, paclitaxel, camptothecin, ellipticine, mithramycin A, etoposide, vinblastine, vincristine, ionomycin and combinations thereof. The heat shocked cancer cells and portions thereof may also be killed by radiation, heat, cold, osmotic shock, pressure, grinding, shearing, ultrasound, drying, freeze spraying, puncturing, starving and combinations thereof, or both my chemical and non-chemical steps. In one embodiment, the cells are killed using natural killer cells.

The present invention also includes a vaccine with killed, allogeneic cancer cells heat-shocked at a temperature of at least 42° C. for at least two hours to form heat shocked allogeneic cancer cells. The cancer vaccine may be made by a method that includes the steps of: incubating at a temperature of at least 42° C. for at least two hours cancer cells; killing the heat shocked cancer cells; and loading antigen presenting cells with cancer cells. Generally, the method and the vaccine will be adapted for administration of the isolated, loaded antigen presenting cells to a patient. In one embodiment, the cancer vaccine for use in a patient may also include one or more at least partially mature antigen presenting cells loaded with heat shocked and killed cancer cells that are not apoptotic.

The vaccines and antigens taught herein may be used in a method of treating a cancer patient by immunizing the patient with a cancer vaccine, including: one or more at least partially mature antigen presenting cells loaded with heat shocked and killed cancer cells. The at least partially mature antigen presenting cells may be autologous and the heat shocked and killed cancer cells may be autologous or allogeneic. For example, the present invention will find particular uses with the heat shocked and killed cancer cells selected from the cells listed hereinbelow and it may be useful to determine and detect the modulation (e.g., upregulation) of the expression of heat shock proteins, e.g., HSP60, HSP90 and gp96, of the cancer cells prior to killing. In certain case, the cancer cells may be transfected to overexpress HSP60, HSP90 and gp96, thereby reducing or eliminating the need to actual heat-shock, however, those cells would fall within the scope of the present invention as these cells would be expressing the one or more heat shock proteins and/or chaperones that help increase the antigenicity of the cancer cells of the present invention.

Another embodiment also includes a method of delivering antigen to dendritic cells in vitro by contacting dendritic cells capable of internalizing one or more antigens for antigen presentation for a time sufficient to allow the one or more antigens to be internalized for presentation to immune cells, wherein the antigen comprises heat-shocked and killed cancer cells. The dendritic cells may be human and the heat-shocked cells may be, e.g., cell lines, cells transformed to express a foreign antigen, tumor cell line, xenogeneic cells, or tumor cells. The heat-shocked cells are selected from the group consisting of the cell lines listed in Table II and combinations thereof and may be killed by chemical treatment, radiation, heat, cold, osmotic shock, pressure, grinding, shearing, ultrasound, drying, freeze spraying, puncturing, starving and combinations thereof. Any of the cells or cell fragments may be contacted with the dendritic cells, e.g., heat-shocked, killed and/or apoptotic cell fragments, blebs, or bodies. Often, the dendritic cells are immature and phagocytic. While the skilled artisan may have to adjust the exact ratios, one example of a common ratio of heat-shocked cells to dendritic cells is about 1-10 heat-shocked cells to about 100 dendritic cells.

After contacting with the antigen, the antigen presenting cells may be further matured, e.g., by exposure to one or more maturation factors for a sufficient time to induce, e.g., the maturation of the dendritic cells. Using dendritic cells as an example, the maturation step may include contacting CD83 negative dendritic cells with at least one maturation factor selected from the group consisting of monocyte conditioned medium that causes CD83 negative dendritic cells to mature so as to express CD83, TNFα, IL-1β, IL-6, PGE₂, IFNα, CD40 ligand, and heat-shocked and killed cells. Other antigen presenting cells may be matured by use of a monocyte conditioned medium; IFNα and at least one other factor selected from the group consisting of IL-1β, IL-6 and TNFα; and heat-shocked cells.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B depict the expression pattern of HSPs after heat shock in melanoma cell lines. As depicted from left to right, melanoma cell lines SKMel28, SKMel24, Me275, Me290 and Colo829 were incubated at 37 degrees C., heat shocked at 42 degrees C. for 2 hours, 4 hours or 8 hours (not shown herein); or treated after heating with 10 μg/ml of betulinic acid (BA) for 24 hours, or treated with 10 μg/ml BA alone for 24 hours and 48 hours. At different time points, cells were harvested and washed twice with cold PBS. Cell pellets were lysed with extraction reagents. From left to right, FIGS. 1A and 1B depict results wherein, FIG. 1A depicts HSP70 expression after heat shock or BA treatment. HSP70 expression was measured with an ELISA kit. FIG. 1B depicts HSP60 expression after heat shock or BA treatment. HSP60 expression was measured with an ELISA kit. The results represent the mean value of two independent studies.

FIG. 2 depicts the experimental design for the study presented in Examples 6 and 7. HLA-A*0201+ monocyte derived dendritic cells were loaded for 3 hours with unheated (cold) or heat treated (hot) melanoma bodies at 1:1 ratio, sorted based on CD 11c expression, matured with sCD40L and used to prime naïve autologous CD8+ T cells in two-week cultures at 10:1 ratio.

FIGS. 3A to 3C depict the priming of CTLs able to kill melanoma cell lines. Representative cell lines were either treated with BA for 48 hours (indicated as “cold”) or heat shocked at 42 degrees C. for 4 hours and then treated with BA for 24 hours (indicated as “hot”). These melanoma bodies were co-cultured with immature MDDCs at 1:1 ratio for 3 hours, and then CD 11c+ MDDCs were sorted and matured with sCD40L (200 nanograms per milliliter) for 24 hours. Autologous naïve CD8 T cells were added at 10:1 ratio with 10 IU/ml of IL-7 for the first week stimulation, and on Day 7, stimulation was repeated as in the first week except replacing IL-7 with IL-2. After the second round stimulation on Day 7, T cells were collected, and the cytotoxic killing activities were detected with a standard 4 hour 51Cr release assay. FIG. 3A depicts 51Cr release from HLA-A*0201+ Me275 melanoma cells and control K562 cells after 4 hours co-culture with primed HLA-A*0201+ CD8+ T cells. CTLun=T cells cultured for two weeks with unloaded DCs, CTLcold=T cells cultured for two weeks with cold Me275 body-loaded DCs, and CTLhot=T cells cultured for two weeks with hot Me275 body-loaded DCs. The results represent the mean and SD of three studies. FIG. 3B depicts 51Cr release from HLA-A*0201+ Me290 melanoma cells, demonstrating that T cells primed by hot Me275 body DCs can cross-kill the Me290 cell line. The T cells are the same as described for FIG. 3A. The results represent the mean and SD of three studies. FIG. 3C depicts the inhibition of specific lysis by pretreatment of target cells with the indicated mAbs, indicating that the cytotoxic activities of T cells primed by hot Me275 bodies were dominantly mediated by MHC class I pathway. The results represent the mean value of two independent studies.

FIG. 4 depicts the cross-priming of CTLs able to kill melanoma cell lines. T cells are primed as described for FIG. 3A-3C, except that the DCs were loaded with HLA-A*0201neg SKMel28 melanoma cells, and the results show the 51Cr release from HLA-A*0201+ SKMel24 melanoma cells (representative of two studies), demonstrating that T cells primed by hot SKMel28 bodies can cross kill SKMel24 cells.

FIGS. 5A to 5D depict the priming of CTLs able to control survival/growth of melanoma cell lines. Naive CD8+ T cells were primed as described in FIG. 3A-3C. EGFP-lentiviral vector transfected melanoma and K562 cell lines were used as targets in the tumor regression assay. As shown in FIG. 5A-5C, after two rounds of stimulations, T cells cultured with unloaded DCs (CTLun) (not shown herein), DCs loaded with cold Me290 bodies (CTLcold), or DCs loaded with hot Me290 bodies (CTLhot) were co-cultured with Me290-EGFP target cells at 20:1 ratio. Co-cultures were harvested at the indicated time points, stained with PE conjugated-anti-CD8 mAb and analyzed by flow cytometry. Values in the upper right indicate the percentage of viable EGFP+ tumor cells. The results are representative of three studies. FIG. 5D depicts T cells primed by DCs loaded with cold Me275 cells (CTLcold) or hot Me275 cells (CTLhot) and co-cultured with Me290-EGFP target cells at 20:1 ratio for 4 hours, 24 hours, 48 hours, or 72 hours. Viable tumor cells were counted by Trypan blue exclusion using light microscopy. The results represent the mean and SD of three studies.

FIGS. 6A to 6F depict the priming of melanoma-specific CTLs: T2 killing assay. In a 4 hour 51Cr release assay, FIG. 6A depicts priming as described in FIG. 3A-3C against HLA-A*0201+ Me290 cells, providing 51Cr release from T2 cells pulsed with either a mix of the four melanoma peptides: MART-1/Melan A, gp100, tyrosinase and MAGE-3 (T2+4P), or with a control PSA peptide (T2+ PSA), or are used unpulsed (T2). The results represent the mean and SD of three studies. FIG. 6B depicts the priming as described in FIG. 4 against HLA-A*0201neg Sk-Mel28 cells, with the read out as given in FIG. 6A. The results represent the mean and SD of three studies. FIG. 6C-6E depict the flow cytometry results for a tumor regression assay of T cells primed by MDDCs loaded with hot apoptotic Me290 bodies co-cultured with T2-EGFP cells (Effector:Target ratio of 30:1), T2-EGFP cells pulsed with PSA peptide (Effector:Target ratio of 30:1), or T2-EGFP cells pulsed with 4 melanoma peptides (gp100, Tyr, MART1 and MAGE3) (Effector:Target ratio of 20:1), respectively, as targets for 0 hours, 4 hours, 24 hours and 48 hours. The results are representative of two studies. According to the FACS data from FIG. 6C-6E, FIG. 6F depicts the growth rates of peptide pulsed T2-EGFP cells calculated by using the following formula: % growth rate=(% EGFP+population at time points/% EGFP+ population at 0 h)×100%. The tumor growth rate at 0 hours was defined as 100%. The results represent the mean value of two studies.

FIGS. 7A to 7C depict the priming of melanoma-specific CTLs: tetramer binding assay, with the priming as described in FIG. 3A-3C against HLA-A*0201+ Me290 cells. As shown in FIG. 7A, tetramer staining was performed on Day 7 after the second stimulation, and 50,000 cells were acquired for each sample. The results indicate the percentage of double positive population (CD8+ Tetramer+) in total CD8 population. As shown in FIG. 7B, primed T cells were re-stimulated once by DCs pulsed with PSA1 (CTLhot-2R/PSA+ DCs) and analyzed 7 days after re-stimulation. As shown in FIG. 7C, primed T cells were re-stimulated once by DCs pulsed with each of the four melanoma peptides (CTLhot-2R/Mel+ DCs) and analyzed 7 days after re-stimulation. The results are representative of two studies.

FIGS. 8A and 8B depict the construction of HSP70 overexpression in a SKMel28 melanoma cell line. FIG. 8A depicts the schematic map of the lentiviral vector RRL-pgk-hsp70-EGFP (used in the tumor regression assay). FIG. 8B depicts HSP70 expression levels in transfected and mock transfected SKMel28 melanoma cell lines. SKMel28, SKMel28/RRL-pkg-EGFP (abbreviated as “SKMel28-EGFP”) and SKMel28/RRL-pkg-HSP70-EGFP (abbreviated as “SKMel28-HSP70-EGFP”) were detected by ELISA and western blotting. The results represent the mean value of three independent studies. Significant HSP70 overexpression was observed (P<0.001 when HSP70 level in SKMel28/RRL-pgk-hsp70-EGFP cell line compared with SKMel28/RRL-pgk-EGFP or SKMel28 cell line).

FIGS. 9A to 9F depict the relative expression data from real-time RT-PCR analysis of melanoma cell lines SkMel28 and Me290 in terms of the mRNA expression of three melanoma antigens, MAGE-B3, MAGE-B4 and MAGE-A8 as described in Example 17. As depicted in FIG. 9A-9F, the melanoma cells were either untreated (“non”); heat treated at 42 degrees C. for 4 hours (“heated 4 hr”); exposed to Actinomycin D, a known transcription inhibitor, during heat treatment at 42 degrees C. for 4 hours (“heat plus AD”); transfected with a control vector EGFP (“EGFP”); or transfected with a vector expression HSP70 (“HSP70”) prior to real-time RT-PCR analysis of the mRNA expression of the melanoma antigens. FIG. 9A depicts SkMel28 cells which were either untreated, heat treated, or transfected as described above prior to real-time RT-PCR analysis of the mRNA expression of MAGE-B3. FIG. 9B depicts Me290 cells which were either untreated or heat treated as described above prior to real-time RT-PCR analysis of the mRNA expression of MAGE-B3. FIG. 9C depicts SkMel28 cells which were untreated, heat treated, or transfected as described above prior to real-time RT-PCR analysis of the mRNA expression of MAGE-B4. FIG. 9D depicts Me290 cells which were either untreated or heat treated as described above prior to real-time RT-PCR analysis of the mRNA expression of MAGE-B4. FIG. 9E depicts SkMel28 cells which were either untreated, heat treated, or transfected as described above prior to real-time RT-PCR analysis of the mRNA expression of MAGE-A8. FIG. 9F depicts Me290 cells which were either untreated or heat treated as described above prior to real-time RT-PCR analysis of the mRNA expression of MAGE-A8.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the terms “antigen-presenting cells” or “APCs” are used to refer to autologous cells that express MHC Class I and/or Class II molecules that present antigens to T cells. Examples of antigen-presenting cells include, e.g., professional or non-professional antigen processing and presenting cells. Examples of professional APCs include, e.g., B cells, whole spleen cells, monocytes, macrophages, dendritic cells, fibroblasts or non-fractionated peripheral blood mononuclear cells (PMBC). Examples of hematopoietic APCs include dendritic cells, B cells and macrophages. Of course, it is understood that one of skill in the art will recognize that other antigen-presenting cells may be useful in the invention and that the invention is not limited to the exemplary cell types described herein.

The APCs may be “loaded” with an antigen that is pulsed, or loaded, with antigenic peptide or recombinant peptide derived from one or more antigens. In one embodiment, a peptide is the antigen and is generally antigenic fragment capable of inducing an immune response that is characterized by the activation of helper T cells, cytolytic T lymphocytes (cytolytic T cells or CTLs) that are directed against a malignancy or infection by a mammal. In one, embodiment the peptide includes one or more peptide fragments of an antigen that are presented by class I MHC or class II MHC molecules. Peptides fragments may be antigens expressed by sarcoma, lymphoma, melanoma or other autologous or heterologous tumors or cancers. Of course, the skilled artisan will recognize that peptides or protein fragments that are one or more fragments of other antigens may used with the present invention and that the invention is not limited to the exemplary peptides, tumor cells, cell clones, cell lines, cell supernatants, cell membranes, and/or antigens that are described herein.

As used herein, the terms “dendritic cell” or “DC” refer to all DCs useful in the present invention, that is, DC is various stages of differentiation, maturation and/or activation. In one embodiment of the present invention, the dendritic cells and responding T cells are derived from healthy volunteers. In another embodiment, the dendritic cells and T cells are derived from patients with cancer or other forms of tumor disease. In yet another embodiment, dendritic cells are used for either autologous or allogeneic application.

As used herein, the term “effective amount” refers to a quantity of an antigen or epitope that is sufficient to induce or amplify an immune response against a tumor antigen, e.g., a tumor cell.

As used herein, the term “vaccine” refers to compositions that affect the course of the disease by causing an effect on cells of the adaptive immune response, namely, B cells and/or T cells. The effect of vaccines can include, for example, induction of cell mediated immunity or alteration of the response of the T cell to its antigen.

As used herein, the term “immunologically effective” refers to an amount of antigen and antigen presenting cells loaded with one or more heat-shocked and/or killed tumor cells that elicit a change in the immune response to prevent or treat a cancer. The amount of antigen-loaded and/or antigen-loaded APCs inserted or reinserted into the patient will vary between individuals depending on many factors. For example, different doses may be required for an effective immune response in a human with a solid tumor or a metastatic tumor.

As used herein, the term “cancer cell” refers to a cell that exhibits an abnormal morphological or proliferative phenotype. The cancer cell may form part of a tumor, in which case it may be defined as a tumor cell. In vitro, cancer cells are characterized by anchorage independent cell growth, loss of contact inhibition and the like, as is known to the skilled artisan. As compared to normal cells, cancer cells may demonstrate abnormal new growth of tissue, e.g., a solid tumor or cells that invade surrounding tissue and metastasize to other body sites. A tumor or cancer “cell line” is generally used to describe those cells that are immortal and that may be grown in vitro. A primary cell is often used to describe a cell that is in primary culture, that is, it is freshly isolated from a patient, tissue or tumor. A cell clone will generally be used to describe a cell that has been isolated or cloned from a single cell and may or may not have been passed in in vitro culture.

As used herein, the term “cancer cell antigen” refers to cells that have been stresses and killed in accordance with the present invention. Briefly, the cancer cells may be treated or stressed such that the cancer cell increases the expression of heat-shock proteins, such as HSP70, HSP60 and GP96, which are a class of proteins that are known to act as molecular chaperones for proteins that are or may be degraded. Generally, these heat-shock proteins will stabilize internal cancer cell antigens such that the cancer cells may include more highly immunogenic cancer cell-specific antigens.

As used herein, the term “contacted” and “exposed”, when applied to an antigen and APC, are used herein to describe the process by which an antigen is placed in direct juxtaposition with the APC. To achieve antigen presentation by the APC, the antigen is provided in an amount effective to “prime” the APCs to express antigen-loaded MHC class I and/or class II antigens on the cell surface.

As used herein, the term “killing” refers to describe causing cell death causes by any number of factors, such as chemical killing using, e.g., betulinic acid, paclitaxel, camptothecin, ellipticine, mithramycin A, etoposide, vinblastine, vincristine, ionomycin and combinations thereof. Any of a number of methods or agents may be used to kill the heat-shocked cancer cells that serve as the antigen of the present invention, e.g., any or a wide variety of radiations (gamma, ultraviolet, microwaves, ultrasound, etc.), heat, cold, osmotic shock, pressure, grinding, shearing, drying, freeze spraying, freeze-drying, vacuum drying, puncturing, starving and combinations thereof. Another type of cell killing or death is referred to commonly as “apoptosis,” which involves the activation of intracellular proteases and nucleases that lead to, for example, cell nucleus involution and nuclear DNA fragmentation. An understanding of the precise mechanisms by which various intracellular molecules interact to achieve cell death is not necessary for practicing the present invention.

As used herein, the phrase “therapeutically effective amount” refers to the amount of antigen-loaded APCs that, when administered to an animal in combination, is effective to kill cancer cells within the animal. The methods and compositions of the present invention are equally suitable for killing a cancer cell or cells both in vitro and in vivo. When the cells to be killed are located within an animal, the present invention may be used in conjunction or as part of a course of treatment that may also include one or more anti-neoplastic agent, e.g., chemical, irradiation, X-rays, UV-irradiation, microwaves, electronic emissions, and the like. The skilled artisan will recognize that the present invention may be used in conjunction with therapeutically effective amount of pharmaceutical composition such a DNA damaging compound, such as, Adriamycin, 5-fluorouracil, etoposide, camptothecin, actinomycin-D, mitomycin C, cisplatin and the like. However, the present invention includes live cells that are going to activate other immune cells that may be affected by the DNA damaging agent. As such, any chemical and/or other course of treatment will generally be timed to maximize the adaptive immune response while at the same time aiding to kill as many cancer cells as possible.

As used herein, the terms “antigen-loaded dendritic cells,” “antigen-pulsed dendritic cells” and the like refer to DCs that have been contacted with an antigen, in this case, cancer cells that have been heat-shocked. Often, dendritic cells require a few hours, or up to a day, to process the antigen for presentation to naive and memory T-cells. It may be desirable to pulse the DC with antigen again after a day or two in order to enhance the uptake and processing of the antigen and/or provide one or more cytokines that will change the level of maturing of the DC. Once a DC has engulfed the antigen (e.g., pre-processed heat-shocked and/or killed cancer cells), it is termed an “antigen-primed DC”. Antigen-priming can be seen in DCs by immunostaining with, e.g., an antibody to the specific cancer cells used for pulsing.

An antigen-loaded or pulsed DC population may be washed, concentrated, and infused directly into the patient as a type of vaccine or treatment against the pathogen or tumor cells from which the antigen originated. Generally, antigen-loaded DC are expected to interact with naive and/or memory T-lymphocytes in vivo, thus causing them to recognize and destroy cells displaying the antigen on their surfaces. In one embodiment, the antigen-loaded DC may even interact with T cells in vitro prior to reintroduction into a patient. The skilled artisan will know how to optimize the number of antigen-loaded DC per infusion, the number and the timing of infusions. For example, it will be common to infuse a patient with 1-2 million antigen-pulsed cells per infusion, but fewer cells may also induce the desired immune response.

The antigen-loaded DCs may be co-cultured with T-lymphocytes to produce antigen-specific T-cells. As used herein, the term “antigen-specific T-cells” refers to T-cells that proliferate upon exposure to the antigen-loaded APCs of the present invention, as well as to develop the ability to attack cells having the specific antigen on their surfaces. Such T-cells, e.g., cytotoxic T-cells, lyse target cells by a number of methods, e.g., releasing toxic enzymes such as granzymes and perforin onto the surface of the target cells or by effecting the entrance of these lytic enzymes into the target cell interior. Generally, cytotoxic T-cells express CD8 on their cell surface. T-cells that express the CD4 antigen CD4, commonly known as “helper” T-cells, can also help promote specific cytotoxic activity and may also be activated by the antigen-loaded APCs of the present invention. In certain embodiments, the cancer cells, the APCs and even the T-cells can be derived from the same donor whose MNC yielded the DC, which can be the patient or an HLA- or obtained from the individual patient that is going to be treated. Alternatively, the cancer cells, the APCs and/or the T-cells can be allogeneic.

The present inventors have found that vaccination of cancer patients with tumor cell antigen loaded antigen presenting cells, e.g., dendritic cells (DCs), can lead to the induction of tumor specific immune responses. However, it has proven difficult to correlate the immune responses with clinical outcomes. Banchereau et al., (2001); and Palucka et al., (2003) reported that 18 HLA-A*0201 patients with stage 1V melanoma were vaccinated with peptide-loaded CD34-DCs, and increased melanoma-specific CD8+ T cell immunity as measured by IFN-γ production (ELISPOT) upon in vitro exposure to melanoma antigen-derived peptides. These studies demonstrated that immune responses correlated with early clinical responses. Furthermore, vaccination with CD34-DCs can elicit melanoma-specific CD8+ memory T cells, which can be expanded in a recall assay, i.e., upon a single restimulation with peptide-pulsed DCs in vitro, where they mature into specific cytotoxic T lymphocytes (CTLs). Disease progression was shown to be associated with the lack of induction of melanoma-specific CD8+ memory T cells. Despite these efforts, improved vaccination strategies are needed to overcome this selective lack of melanoma-specific immunity in the clinic.

For example, DCs have been shown to act as immune reservoirs or adjuvants in healthy volunteers and in stage 1V melanoma patients (Nestle et al., 1998; Dhodapkar et al., 1999; Thurner et al., 1999; and Dhodapkar et al., 2000). To date, only limited clinical responses have been reported, which may be due to the choice of the immunizing epitope or targeting of a single epitope. Indeed, the use of multiple tumor antigens facilitates activation/induction of T cells with multiple specificities, which might be able to better control the disease and prevent tumor escape. In this regard, several systems have been employed to load DCs with tumor-associated antigen (TAA) (Gilboa, E. 1999). Loading MHC class I molecules with peptides derived from defined antigens is most commonly used, and is also applied to recently identified MHC class II helper epitopes (Wang et al., 1999; and Kierstead, et al., 2001).

Although important for “proof of concept” studies, the use of peptides has limitations coming from: (i) their restriction to a given HLA type; (ii) the limited number of defined TAA; and (iii) the induction of a restricted repertoire of T cell clones, thus limiting the ability of the immune system to control tumor antigen variation. Alternative strategies that provide both MHC class I and class II epitopes and lead to a diverse immune response involving many clones of CD4+ T cells and CTLs are needed. Reported strategies involve use of recombinant proteins, exosomes (Zitvogel, et al., 1998), viral vectors (Ribas, et al., 2002), plasmid DNA or RNA transfection (Boczkowski, et al., 1996; Ashley et al., 1997; and Heiser, et al., 2002), immune complexes (Regnault, 1999) and, more recently, antibodies against DC surface molecules (Gilboa, E. 1999; and Fong, et al., 2000).

Yet another way to diversify immune response is to exploit the capacity of DCs to present peptides from phagocytized apoptotic tumor cells, or so called cross-priming (Albert et al., 1998a; Albert et al., 1998b; Nouri-Shirazi et al., 2000; Berard et al., 2000; and Labarriere et al., 2002). The present inventors have found that introducing whole antigen into DCs allows the DCs to select and tailor peptides for presentation to T cells, and thus, circumvents the need to identify tumor-specific peptides with known MHC restrictions. It has been shown that DCs loaded with killed allogeneic melanoma cells can cross-prime naïve CD8+ T cells to differentiate into melanoma-specific CTLs (Berard, et al., 2000). DCs loaded with killed allogeneic melanoma or prostate cancer cell lines prime naïve CD8 T cells against shared tumor antigens (Nouri-Shirazi, et al., 2000; and Berard, et al., 2000). Yet, T cells require several rounds of stimulation for the tumor specific responses to be established. Therefore, it is important to identify and developed compositions and methods for increasing tumor or cancer cell immunogenicity.

The present inventors recognized that heat shock proteins (HSPs) constitute molecular chaperones for the transit of polypeptides from their generation to, e.g., their binding to MHC class I in the endoplasmic reticulum (ER) (Basu, et al., 2000; and Frydman, J. 2001). HSP70, HSP60 and GP96 have been established recently as immune adjuvants for cross-priming with antigenic proteins or peptides (Srivastava, et al., 1994; and Srivastava, P., 2002). In this process, reconstituted hsp70-peptide complex or gp96-peptide complex are internalized by antigen-presenting cells (APC) through receptor-mediated endocytosis via CD91 (Basu, et al., 2001), CD40 (Becker, et al., 2002), LOX-1 (Delneste, et al., 2002), or TLR2/4 (Asea, et al., 2002). HSP:peptide complexes have been used as vaccines (U.S. Pat. No. 6,468,540; and Noessner, et al. 2002. “Tumor-derived heat shock protein 70 peptide complexes are cross-presented by human dendritic cells,” J Immunol 169:5424-5432). An HSP70:peptide complex may be purified from the patient's tumor cells and administered to the patient (U.S. Pat. No. 6,468,540). It has been determined that HSP70:peptide complexes are able to bind to antigen presenting cells and activate cytotoxic T cells (Castelli, et al. 2001. “Human heat shock protein 70 peptide complexes specifically activate antimelanoma T cells,” Can Res 61:222-227; and Noessner, et al. 2002. J Immunol 169:5424-5432). HSP70 has also been used to stimulate dendritic cells to mature (U.S. Patent Application No. 20020127718).

More particularly, the present invention includes antigen presenting cells, e.g., Dendritic cells (DCs), that are loaded with stressed and/or heat shocked killed tumor cells, or killed tumor cells expressing heat shock proteins, and the methods for making such antigen-presenting cells are described herein. These loaded DCs are useful to induce both prophylactic immune responses and therapeutic immune responses in humans and animals. In particular, such loaded DCs are useful in the management of cancer and infectious diseases.

In one embodiment, the present invention includes a dendritic cell (DC) vaccine for the treatment of melanoma that integrates the following immunogenic phenomena: (i) the capacity of DCs to cross-prime melanoma-specific CTLs, (ii) the advantage of using killed tumor cells as the source of antigens, (iii) the favorable roles of HSPs in peptide protection and transport, and (iv) as demonstrated herein, the up-regulation of tumor antigen expression by heat shock. In one embodiment, the DC vaccines of the present invention include DCs loaded with heat shocked killed tumor cells, wherein the DCs are capable of cross-priming antigen-specific cytotoxic T lymphocytes. In another embodiment, the DC vaccines of the present invention include DCs loaded with killed tumor cells that have been induced to overexpress HSPs, wherein the DCs are capable of cross-priming antigen-specific cytotoxic T lymphocytes. Unless specified otherwise, it is to be understood that when reference is made herein to DC vaccines comprising DCs loaded with heat shocked killed tumor cells, a DC vaccine comprising DCs loaded with killed tumor cells transfected or induced to overexpress HSPs may also be used.

DCs useful in the present invention include dendritic cells at various differentiation stages (precursors, immature dendritic cells and mature dendritic cells), dendritic cells derived from blood precursors including but not limited to monocytes, dendritic cells derived from CD34-hematopoietic progenitor cells, subsets of dendritic cells such as Langerhans cells, interstitial DCs and lymphoid DCs. In one embodiment, the dendritic cells are monocyte derived dendritic cells (MDDCs), e.g., the DCs are of human origin.

Vaccine Regimens and Dosage. Any vaccination regimen may be followed for use with the present invention, however, the following exemplary regimes have been used to great effect as will be known to those of skill in the art. One or more vaccination may be preceded or followed by the administration of additional peptide-pulsed APC by intervals ranging from seconds to hours to days to even weeks. In one embodiment, the cell debris-pulsed APCs and one or more lymphokines and/or cytokines are administered separately to the patient. Often, a significant period of time (1, 2, 3 or 4 weeks) is selected between the time of each immunization, such that the combination and/or overlap of two antigen-pulsed APCs exerts an advantageous effect on the recipient.

For example, the administration of peptide-pulsed APC will be desired in certain circumstances in combination with one or more lymphokines that drives, e.g., the T cell immune response from a Th1 to a Th2-type response, or vise versa. Various combinations may be employed, e.g., where peptide-pulsed APC is “A” and the lymphokine is “B”:

| | | | |
|---|---|---|---|
| A/B/B | B/A/A | A/A/B | |
| A/B/A | B/A/B | B/B/A | |
| B/B/B/A | B/B/A/B | B/A/B/A | B/A/A/B |
| A/A/B/B | A/B/A/B | A/B/B/A | B/B/A/A |
| A/A/A/B | B/A/A/A | A/B/A/A | |
| B/A/B/B | A/A/B/A | A/B/B/B | |

Effective tumor killing may be measured before, during and/or after the initiation of the vaccination regimen. To achieve tumor cell killing, the antigen-loaded APCs are delivered to a patient in a combined amount effective to kill the tumor cells. These treatment cycles can be repeated multiple times, or delivered only once. The skilled artisan that various factors are well known to influence patient response to vaccination, including, e.g., species, age, weight, gender, health, pregnancy, addictions, allergies, ethnic origin, prior medical conditions, current medical condition, treatment with anti-inflammatories, chemotherapy and length of treatment. Thus, the skilled artisan understands the need to individualize dosage(s) to each patient and the various parameters that may easily be varied to achieve the optimal immune response, whether its cell killing (e.g., against cancer) or the reduction of an untoward immune response (e.g., cachexia). The skilled artisan may also consider the condition that is to be treated prior to selecting the appropriate dosage. For example, a vaccination dosage that is appropriate for the treatment of a cancer, may not be the desired dosage for subsequent surveillance therapy designed to prevent the recurrence of the cancer.

Vaccinations may be administered intravenously, intra-arterially, intratumorally, parenterally, intraperitoneally, intramuscular, under the kidney capsule, intraocularly, intraosseally, intravaginally, rectally, epidural, intradural, and the like. Often, the most common routes of vaccination are subcutaneous (SC), intravenous (IV), intrarterial, and intraperitoneal (IP). To the extent that the vaccines are compatible with buffers and/or pharmacologically acceptable salts these can be prepared in aqueous solution suitably mixed with one or more additives. Under ordinary conditions of storage and use, these preparations may include limited amounts of a preservative and/or an antibiotic to prevent the growth of microorganisms.

The pharmaceutical forms suitable for injectable use include sterile aqueous solutions or dispersions. In all cases the form must be sterile and must be fluid to the extent that easy syringability exists. The storage conditions, if any, must be compatible with the delivery of stable DCs under the conditions of manufacture and storage and must be preserved against the contaminating action of microorganisms, such as bacteria and fungi. In most cases, it may be common to include one or isotonic agents, for example, sugars or sodium chloride. Prolonged absorption of the antigen with the APCs may be brought about by the use in the antigens of delaying absorption, for example, aluminum monostearate, calcium phosphate, and gelatin.

Sterile injectable solutions are prepared by incorporating the active compounds in the required amount in the appropriate solvent with various other ingredients enumerated above that may have been, e.g., filtered sterilization. Generally, dispersions are prepared by incorporating the various sterilized active ingredients into a sterile vehicle that includes a basic dispersion medium and the required other ingredients from those enumerated above. In the case of sterile powders for the preparation of antigens, the antigens may be pre-prepared and vacuum-dried, freeze-dried and/or freeze-sprayed to yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the cancer antigen, the agent may be used as part of the vaccine production process.

As used herein, the phrase “under conditions effective to allow protein complex formation” refers to those conditions and amounts of a heat killed, killed or otherwise processed tumor cells, tumor cell debris, processed tumor antigens, processed tumor cells, heat-killed tumor cells and/or antigens that are needed to “load” the MHC of an APC, e.g., a dendritic cell. As used herein, the term “suitable” for antigen loading are those conditions that permit a DC to contact, process and present one or more tumor antigens on MHC, whether intracellular or on the cell surface. Based on the present disclosure and the examples herein, the skilled artisan will know the incubation, temperature and time period sufficient to allow effective binding, processing and loading. Incubation steps are typically from between about 1 to 2 to 4 hours, at temperatures of between about 25 degrees to 37 degrees C. (or higher) and/or may be overnight at about 4 degrees C. and the like.

In one example of the present invention, the APCs are DCs loaded with dead or dying tumor cells (referred to herein as “killed tumor cells”), including but not limited to tumor cell lines and isolated autologous or allogeneic tumor cells. It is foreseeable that any tumor or cancer cells isolated from a patient or available from other sources may be used in an embodiment of the present invention. While the examples disclose use of melanoma cell lines, it is contemplated that an embodiment of the present invention may be used in the treatment of other cancers, and the type of cancer treatable by an embodiment of the present invention depends upon the type of cancer cells used to load the dendritic cells.

In the examples presented herein, death of the tumor cells is accomplished by treatment with betulinic acid (BA). BA is a particularly active agent against melanoma which induces mitochondria-dependent death through activation of caspase-8 and caspase-3. Although betulinic acid (BA) is used to induce apoptosis or cell death of the melanoma cell lines used in the examples presented herein, other cell death inducing agents may be used in place of BA in an embodiment of the present invention. Other cell death inducing agents include but are not limited to betulinic acid, paclitaxel, camptothecin, ellipticine, mithramycin A, etoposide, vinblastine and vincristine.

The DC vaccines of the present invention comprise DCs loaded with killed tumor cells which have either been treated to induce the expression of heat shock proteins (HSP) or have been transfected to overexpress HSPs. In one preferred embodiment, the DC vaccine comprises monocyte derived dendritic cells (MDDCs) loaded with killed tumor cells that were previously incubated at least 42 degrees C. (referred to herein as “heat shocked”) for at least 4 hours to induce HSP expression. In another preferred embodiment, the DC vaccine comprises MDDCs loaded with tumor bodies having been transfected with a vector comprising an HSP overexpression gene, for example, the RRL-pgk-HSP70-EGFP lentiviral vector as described herein. Other HSPs that can be used in the present invention include but are not limited to HSP60, HSP90 and gp96. Although 42 degrees C. is used in the examples for heat shocking the tumor bodies or cells, any temperature sufficient to increase expression of HSPs yet retain the function of the HSPs may be used in an embodiment of the present invention (e.g., approximately 39-55 degrees C.). Other methods of increasing expression of HSPs in cells include but are not limited to cold temperature, glucose deprivation or oxygen deprivation, exposure to drugs that alter cell metabolism, exposure to cytotoxic drugs and other stress signals. Moreover, while the examples show heat shocking for 2, 4 or 8 hours, any period of time sufficient to increase the expression of HSP70 or other HSP may be used in an embodiment of the present invention.

According to the present invention, the DCs loaded with killed tumor cells are capable of eliciting cytotoxic cells (CTLs) which are able to kill tumor cells as well as target cells loaded with tumor associated antigen derived peptides. The cytotoxic cells include but are not limited to CD8 T cells, CD4 T cells, natural killer cells, and natural killer T cells. It is to be understood hereinafter that unless stated otherwise, reference to cytotoxic T cells refers to one or more of the cytotoxic cells. According to the present invention, CTLs are prepared by co-culturing the cytotoxic cells, such as CD8+ T cells, with DCs loaded with heat shocked killed tumor cells. In one method, heat shocked killed tumor cells are co-incubated with MDDCs at a 1:1 ratio at 37 degrees C.; after 3 hours co-incubation, cells are suspended with 0.05% trypsin/0.02% EDTA PBS solution for 5 minutes to disrupt the cell-cell binding; CD11c+DCs are sorted, matured with sCD40L (200 nanograms per milliliter) for 24 hours and then employed to prime the cytotoxic cells. According to the present invention, any incubation temperature and any amount of time of co-culture of the loaded dendritic cells that allows uptake of HSP:tumor antigen complexes by the DCs can be used as will be known to the skilled immunologist.

DCs loaded with heat shocked killed tumor cells can also prime naïve T cells to differentiate into effector cells able to recognize either specific antigens or multiple and/or shared tumor antigens that are expressed either on the tumor cells that are used to load the dendritic cells and/or on other tumor cells. This cross-priming against multiple antigens shared between different cells, for instance tumor cells, is important to elicit broad immune responses. In the present invention, the CTL elicited by DC loaded with cell bodies from one specific allogeneic tumor source can be used to provide a killing effect on other tumors. For example, CTL elicited by DC loaded with killed tumor cells derived from a specific allogeneic melanoma carcinoma cell line such as Me275 can kill other tumor cells lines such as Me290.

The methods of the present invention also include the treatment of a patient a tumor by treating the patient with the antigen of the present invention in an appropriate vector for vaccination, e.g., autologous dendritic cells loaded with heat shocked, killed tumor cells. In one embodiment, the patient is treated with DCs loaded with heat shocked, killed tumor cells from the same patient. In another embodiment, the patient is treated with DCs loaded with killed tumor cells previously induced to overexpress HSPs. In another embodiment, the patient is treated with autologous T cells primed by autologous or allogeneic dendritic cells loaded with autologous or allogeneic heat shocked killed tumor cells. In yet another embodiment, the patient is treated with autologous T cells primed by autologous or allogeneic dendritic cells loaded with killed tumor cells previously heat shocked and/or transfected to overexpress HSPs. A similar protocol would be followed for prophylactic treatment. The route of vaccine administration in the present invention includes but is not limited to subcutaneous, intracutaneous, in the kidney capsule, intraoptical or intradermal injection.

The frequency of vaccine administration may be individualized based on evaluating blood immune responses after the first vaccination. The presence of immune responses at such an early stage identifies patients that require less frequent vaccination, for example on a monthly basis. The absence of immune responses at this stage identifies patients that require more frequent vaccination, for example every other week. In the present invention, patients should be vaccinated for a life-time or until progression of malignancy. Similar protocol would be followed for prophylactic treatment.

In the present invention, the comprehensive evaluation of elicited immunity against tumor antigens can be determined by any method known in the art. For example, the immunogenicity of the DCs of the present invention can be measured by several parameters of CD8+ T cell cross-priming including the following methods: (i) the number of stimulations with loaded DCs needed for naïve CD8+ T cell differentiation, (ii) killing of HLA-A*0201 melanoma cells in a standard 4 hour 51Cr release assay, (iii) the capacity to prevent tumor growth in vitro in a tumor regression assay, (iv) killing of melanoma peptide-pulsed T2 cells, and (v) the binding of melanoma tetramers.

The compositions and methods of use of the present invention are further illustrated in detail in the examples provided below, but these examples are not to be construed to limit the scope of the invention in any way. While these examples describe the invention, it is understood that modifications to the compositions and methods are well within the skill of one in the art, and such modifications are considered within the scope of the invention.

### EXAMPLE 1

Cell Lines and Cell Culture. Human melanoma cell lines: HLA-A*0201+ Me275 and HLA-A*0201+ Me290 lines were established at the Ludwig Cancer Institute in Lausanne, and were a kind gift of Drs. J-C. Cerottini and D. Rimoldi. Breast cancer cell line MCF-7 (HLA-A2+) (ATCC No. HTB-22) and T2 (HLA-A2+) (ATCC No. CRL-1922) were from the American Type Culture Collection (ATCC; Manassas, Va.). K562 (ATCC No. CCL-243) is a multipotential, hematopoietic, malignant cell line. Colo829 (ATCC No. CRL-1974) is a malignant melanoma cell line. HLA-A*0201neg SKMel28 and HLA-A*0201+ SKMel24 are malignant melanoma cell lines obtained from ATCC. All these cell lines were maintained in complete culture medium (CM) consisting of RPMI 1640 (GIBCO BRL), 1% L-glutamine, 1% penicillin/streptomycin and 10% heat-inactivated fetal calf serum (FCS). For T cell cultures, FCS was replaced by 10% heat-inactivated human AB serum.

### EXAMPLE 2

Generation of EGFP+ Cell Lines. The HLA-A201+ allogeneic cell lines T2, K562, Me275, Me290 and MCF7 were transfected with the lentiviral vector pHREF1α-EGFP (kindly provided by Dr. Patrice Mannoni), which encode the EGFP placed under the control of the Elongation Factor 1α promoter. Transduction of cell lines was performed at a multiplicity of infection (MOI) of 15 for 6 hours with 8 micrograms per milliliter of polybrene (Sigma-Aldrich, St. Louis Mo.) at 37 degrees C. in a 5% CO₂ incubator. Fresh media was then added, and culture was resumed. At Day 2 post-transduction, EGFP expression was monitored by flow cytometry. Cells were expanded and sorted to a purity of >95% EGFP+ cells. They were counted and resuspended at 5.104/ml in cRPMI+10% AB.

### EXAMPLE 3

Reagents and Peptides. The recombinant human cytokines used were GM-CSF (Immunex), soluble CD40 ligand (sCD40L), IL-2, IL-7 and IL-4 (R&D Systems, Minneapolis, Minn.). Betulinic acid (BA) and DNA dye 7-aminoactinomycin D (7-AAD) were purchased from Sigma-Aldrich (St. Louis, Mo.). Peptides: gp100₂₀₉₋₂₁₇ (IMDQVPFSV; SEQ ID NO:1), tyrosinase₃₆₈₋₃₇₆ (YMDGTMSQV; SEQ ID NO:2), MART1₂₇₋₃₅ (AAGIGILTV; SEQ ID NO:3), MAGE3₂₇₁₋₂₇₉ (FLWGPRALV; SEQ ID NO:4) and PSA1₁₄₁₋₁₅₀ (FLTPKKLQCV; SEQ ID NO:5) were synthesized by Bio-Synthesis (Lewisville, Tex.). Lyophilized peptides were dissolved in DMSO, diluted to 1 milligram per milliliter in apyrogen water, and stored at −80 degrees C.

### EXAMPLE 4

Preparation of Heat Shocked Killed Melanoma Cells. Melanoma Cell lines were plated into a 250 ml flask at 3×10⁵ cells per milliliter concentration in CM. Melanoma cells that were both heat shocked and killed were prepared as follows. After 24-hour culture at 37 degrees C., the cells were moved to a 42 degrees C. incubator for 2 hours or 4 hours. The cells were then incubated at 37 degrees C. with the addition of 10 micrograms per milliliter of BA, a compound reported to induce apoptosis or cell death, and incubated for an additional 24 hours. Hereinafter, these cells are referred to as “hot melanoma bodies.” CD8⁺ T cells primed with DCs loaded with hot melanoma bodies are hereinafter referred to as hot “CTL^{hot}.”

For melanoma cells that were killed but not heat shocked, the cells were treated with 10 micrograms per milliliter of BA for either 24 or 48 hours at 37 degrees C. Hereinafter, these cells are referred to as “cold melanoma bodies.” CD8⁺ T cells primed with DCs loaded with cold melanoma bodies are hereinafter referred to as “CTL^{cold}.” For melanoma cells that were heat shocked but not treated with BA, the cells were moved to a 42 degrees C. incubator for 2 hours or 4 hours and then incubated for an additional 24 hours at 37 degrees C. without the addition of BA. Hereinafter, these cells are referred to as “heat-shocked melanoma cells”.

CD8⁺ T cells primed with unloaded DCs were used as controls in many experiments presented herein. These controls are referred to as “CTL^{un}.” APC-conjugated annexin-V and propidium iodide (PI) staining were used to detect the percentages of apoptosis of tumor cells under different conditions.

### EXAMPLE 5

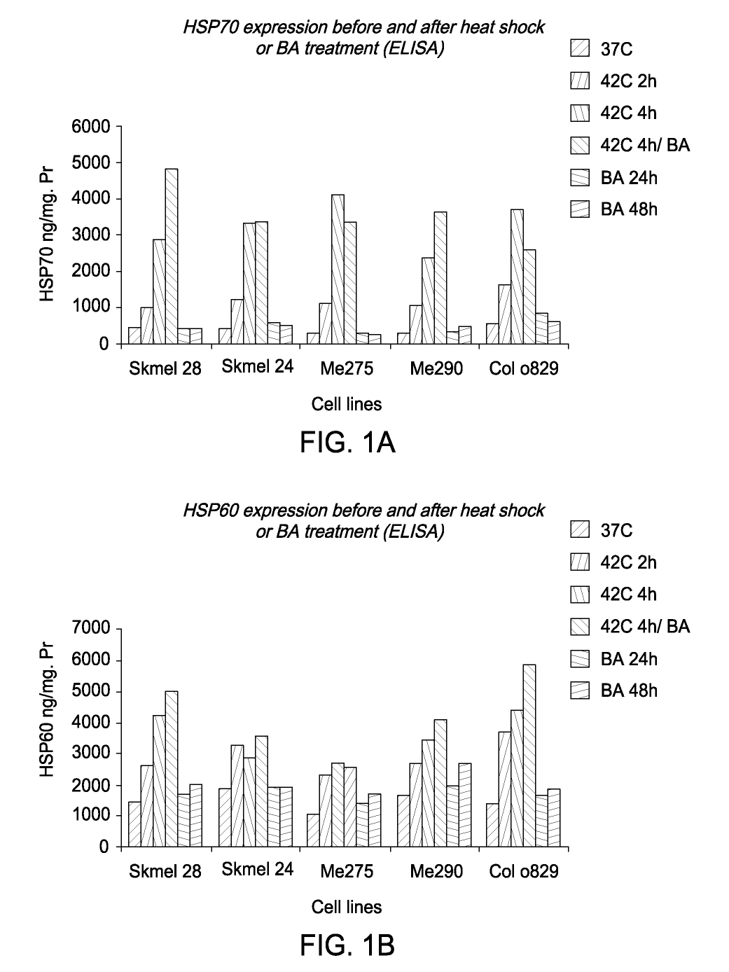

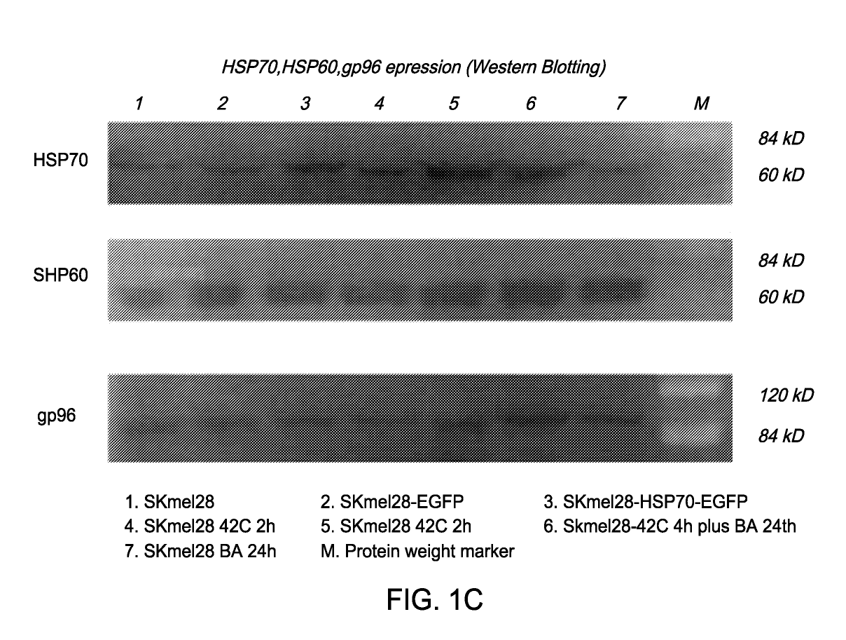

Determination of HSP Expression. Melanoma cell lines SKMel28, SKMel24, Me275, Me290 and Colo829 were either heat shocked, heat-shocked-plus-BA-treated, or BA-treated. Representative cells were collected and washed twice with cold phosphate buffered saline (PBS). The cell pellets were resuspended with an appropriate volume of lysis buffer supplemented with protease inhibitor cocktail (0.1 mM PMSF, 1 microgram per milliliter leupeptin, 1 microgram per milliliter aprotinin, and 1 microgram per milliliter pepstatin) and incubated on ice for 30 minutes with occasional mixing until the cell suspension was homogeneous and no clumps were visible. The cell lysate was centrifuged at 12,000 rpm for 20 minutes at 4 degrees C. HSP60 and HSP70 levels in the supernatant were detected by an ELISA kit (Stressgenes, Canada) (FIGS. 1A and 1B). The total protein in cell supernatant was examined with Micro BSA™ protein Assay reagent Kit (Pierce Biotechnology, Inc. Rockford, Ill.). HSP60 or HSP70 concentrations in cell lysates were defined as nanograms HSP per milligram of protein (ng/mg Pr) in the supernatant. As shown in FIG. 1A, the HSP70 expression in each melanoma cell line was greatly increased in 4-hour heat shocked melanoma cells and heat-shocked-plus-BA-treated cells (hot melanoma bodies). In FIG. 1B, an increase in HSP60 expression is indicated for 2-hour and 4-hour heat shocked melanoma cells and heat-shocked-plus-BA-treated cells (hot melanoma bodies).

Western blotting was also used to measure HSP70, HSP60 and GP96 expression patterns in a SKMel28 cell line for heat shocked cells (2 hours or 4 hours), heat-shock-plus-BA-treated cells (hot melanoma bodies) and BA-treated cells (cold melanoma bodies) (24 hours). Each cell lysate containing 30 micrograms total protein was loaded and separated in 8% SDS-polyacrylamide gel, and transferred onto PVDF membranes (Novex, San Diego). The membranes were blocked overnight at 4 degrees C. by using Super Blocking buffer (Pierce Biotechnology, Inc.) and incubated with 1 micrograms per milliliter of mouse anti-human HSP60 (SPA806), HSP70 (SPA810), or gp96 (SPA851) monoclonal antibodies (Stressgene) for 2 hours at room temperature. After washing membranes with PBS-T buffer, HRP-conjugated goat anti-mouse IgG was added for 1 hour incubation, and the protein blots were revealed with Fluoro Blot™ peroxidase substrate (Pierce Biotechnology, Inc.). Results showed increases in HSP60, HSP70 and GP96 expression for 4-hour heat shocked melanoma cells and heat-shocked-plus-BA-treated cells (hot melanoma bodies) (data not shown).

### EXAMPLE 6

Monocyte-Derived Dendritic Cell Generation and Antigen Loading. PBMCs from HLA-A*0201⁺ healthy donors or G-CSF mobilized HLA-A*0201⁺ healthy donors were plated into 6-well plates and allowed to adhere for 2 hours at 37 degrees C. The non-adherent cells were removed, and the adherent cells were cultured in CM supplemented with GM-CSF (100 nanograms per milliliter) and IL-4 (25 nanograms per milliliter). DCs were fed by adding fresh GM-CSF and IL-4 medium every 2 days. On Day 5, immature monocyte derived dendritic cells (MDDCs) were harvested and washed with PBS, then labeled with CD11c-APC for 30 minutes at 4 degrees C.

Killed tumor cells were co-incubated with labeled MDDCs at a 1:1 ratio at 37 degrees C. After 3 hours co-incubation, cells were suspended with 0.05% trypsin/0.02% EDTA PBS solution for 5 minutes to disrupt the cell-cell binding. CD11c⁺ DCs were sorted, matured with sCD40L (200 nanograms per milliliter) for 24 hours and employed to prime naïve CD8⁺ T cells.

Confirmation of phagocytosis of tumor bodies by dendritic cells. To confirm that the killed tumor cells were captured by the immature MDDC, the killed tumor cells were stained with DNA-specific dye, 7AAD, for 30 minutes at 4 degrees C., and then were co-incubated with CD11c-APC labeled immature MDDCs at different ratios (3:1, 1:1 or 1:3) at 4 degrees C. or 37 degrees C. After 2 hour's culture, phagocytosis of the killed tumor bodies by the DCs was demonstrated by FACS as the percentage of double-positive DCs (i.e., CD11c⁺7AAD⁺) in total CD11c⁺DC population (data not shown). The internalization of killed tumor bodies by DCs was also confirmed with confocal microscopy. Briefly, the co-culture mixture of DCs and tumor bodies was mounted to poly-lysine-coated slides (Baxter Diagnostics, Deerfield, Ill.), fixed with 4% paraformaldehyde, and permeabilized with 0.5% saponin/0.2% BSA/0.2% gelatin solution. gp100 monoclonal antibody (NKI/beteb, Biodesign International, Saco, Me.) and CD1a-FITC-conjugated mAb were used to respectively identify tumor bodies and DCs. The gp100 staining occurring in the cytoplasm of CD1a+ labeled MDDCs was an indication that the killed tumor bodies were captured by MDDCs (data not shown).

### EXAMPLE 7

Naïve CD8+ T Cell Purification and Priming. The capacity of monocyte derived dendritic cells (MDDCs) loaded with heat shocked killed tumor cells to prime naïve CD8+ T lymphocytes was examined.

CD8⁺ T cells were enriched from PBMCs of HLA-A*0201⁺ health donors by depletion of other cells using mouse anti-human-CD4, CD14, CD16, CD56, CD19, and glycophorin A microbeads (Miltenyi Biotec, Inc., Auburn, Calif.). The depletion performance was carried out by the AutoMACS system (Miltenyi Biotec, Inc.). The enriched CD8⁺ T cells were stained with anti-CD27-FITC, CD45RA-PE, CD8-QR, and CD45RO-APC and sorted as CD8⁺ CD45RA⁺CD27⁺CD45RO⁻ naïve T cells (>95% purity). Naive T cells were co-cultured with matured unloaded DCs or loaded DCs at a 10:1 ratio supplemented with 10 IU/ml of IL-7 in the first week, and IL-2 in the 2^{nd} week. T cells were restimulated at Day 7.

### EXAMPLE 8

⁵¹Cr Release Assay. Target cells were labeled with Na⁵¹CrO₄ for 1 hour at 37 degrees C. T2 cells were pulsed with 4 melanoma peptides (gp100, Tyr, MART1 and MAGE3) for 3 hours before labeling. A 4-hour-standard killing assay was performed as described earlier (Paczesny et al., 2004). Briefly, effector cells (30×10³/well) were plated in 96-well round-bottom plates along with the ⁵¹Cr labeled target cells. After 4 hours, supernatants were harvested using a harvesting frame and released chromium-labeled protein was measured using γ-counter (Packard Instruments Co, Meriden, Conn., US). Percentage of antigen-specific lysis was then determined.

For blocking, ⁵¹Cr-labeled targets were co-incubated with 10 micrograms per milliliter of purified mouse anti-human HLA-ABC mAb (clone W6/32, DAKO, Carpinteria, Calif.) or HLA-DR mAb (Clone G46-6, BD Biosciences Pharmingen, San Diego, Calif.) or matched mouse IgG isotypes (Clone G155-178 or G46-6, BD Biosciences Pharmingen) in a 96 well plate for 30 minutes, and then T cells were added for the 4-hour-standard killing assay. The mean of triplicate wells for each sample was calculated, and the percentage of specific ⁵¹Cr release was determined according to the following formula:

$$\% specific {}^{51} Cr release = 100 \times \frac{( \begin{matrix}experimental {}^{51} Cr release - \\ spontaneous release\end{matrix} )}{( \begin{matrix}maximum {}^{51} Cr release - \\ spontaneous release\end{matrix} )}$$

### EXAMPLE 9

Tumor Regression Assay. Tumor cell lines were transfected with lentiviral vector encoding EGFP as previously described (Paczesny et al., 2004) and briefly presented in Example 2. Cell lines were suspended at a concentration of 5×10⁴ cells/ml with RPMI 1640 medium containing 10% AB serum. Primed T cell lines were suspended at 10⁶ cells/ml. Targets and T cells were co-incubated in a 96 well-U-bottom plate for 0 hours, 4 hours, 24 hours, 48 hours and 72 hours in 200 microliters of total volume. At each time point, the cell mixture was harvested and treated with 0.05% trypsin/0.02% EDTA PBS solution for 5 minutes. Cell pellets were stained with PE-conjugated CD8 mAb and analyzed by using FACS Calibur™ (Becton Dickinson, San Jose, Calif.). For each sample, 50,000 cells were acquired. The percentage of EGFP+ population (gate R1) in total population (no gate) was quantized by CELLQuest™ software (Becton-Dickinson). The tumor growth rates were calculated by using the following formula:

$$\% growth rate = \frac{\% EGFP + population at given time point}{\% EGFP + population at 0 hour} \times 100 \%$$

The tumor growth rate at 0 hour was defined as 100%. To count the live cells using light microscopy, Trypan blue exclusion was used.

### EXAMPLE 10

Tetramer Staining. The iTAgTMMHC Tetramers: HLA-A0201/gp100 (IMDQVPFSV), HLA-A0201/MAGE3 (FLWGPRALV), HLA-A0201/Tyrosinase (YMDGTMSQV), and HLA-A0201/MART1 (ELAGIGILTV) peptide tetramers were purchased from Beckman-Coulter. Primed T cell lines were stained with PE-conjugated tetramer for 30 minutes and with PerCP- or FITC-conjugated anti-CD8 mAb for another 30 minutes at room temperature. Cells were analyzed by flow cytometry.

### EXAMPLE 11

Recall Assay. CD8 T cells after two stimulations with melanoma body-loaded DCs were plated with peptide pulsed autologous DCs at 10:1 ratio. The T cells were analyzed after 7 days of culture for the frequency of melanoma-specific CD8⁺ T cells.

### EXAMPLE 12

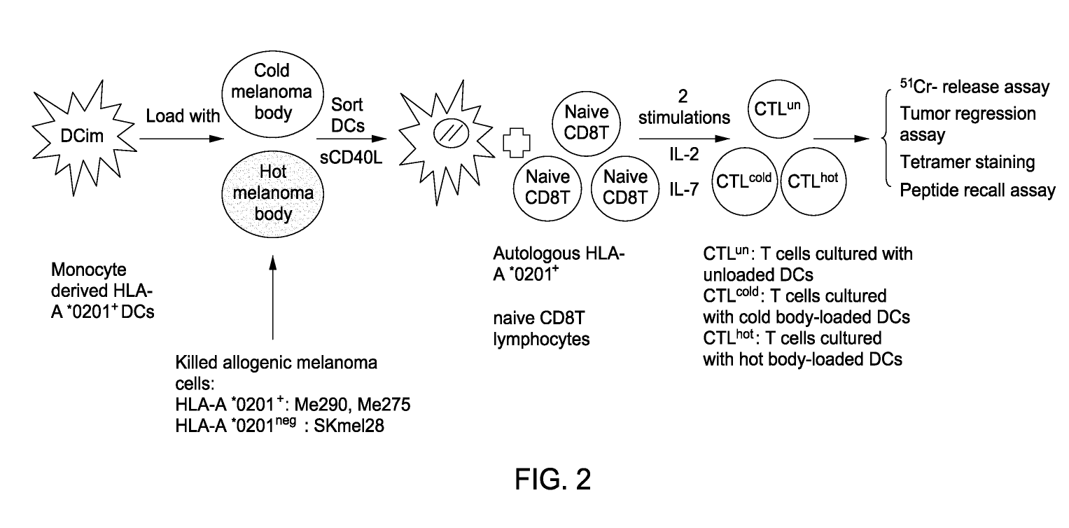

Cross-Priming of Melanoma-Specific CTLs. As illustrated in FIG. 2, immature DCs were generated from monocytes of HLA-A*0201⁺ healthy volunteers by culturing with GM-CSF and IL-4. Melanoma cell lines were incubated for 4 hours at 42 degrees C. (heat shock) prior to killing. Melanoma bodies were generated from either unheated (cold melanoma bodies) or heated (hot melanoma bodies) melanoma cells by 24-hour treatment with betulinic acid (BA) as given in Example 4 and as previously described (Berard et al., 2000). These killed tumor cells were co-cultured with immature MDDCs at 1:1 ratio for 3 hours to generate DCs loaded with cold melanoma bodies and DCs loaded with hot melanoma bodies as described in Example 6. Unloaded DCs, DCs loaded with cold melanoma bodies, and DCs loaded with hot melanoma bodies were sorted and cultured with purified CD8⁺ CD45RA⁺CD27⁺CD45RO⁻ naïve T cells. DC/T cell (1:10 ratio) co-cultures were supplemented with soluble CD40 ligand (200 nanograms per milliliter), IL-7 (10 U/ml, throughout the culture) and IL-2 in the second week (10 U/ml). T cells were restimulated with antigen loaded DCs once unless otherwise indicated. On Day 7 after the second round of stimulation, the cells were harvested to detect the cytotoxic killing activity as detected with a 4-hour ⁵¹Cr release assay as well as the frequency of melanoma-specific effector T cells. This process as presented in Example 7 resulted in T cells primed with unloaded DCs (CTL^{un}), DCs loaded with cold melanoma bodies (CTL^{cold}), and DCs loaded with hot melanoma bodies (CTL^{hot}).

### EXAMPLE 13

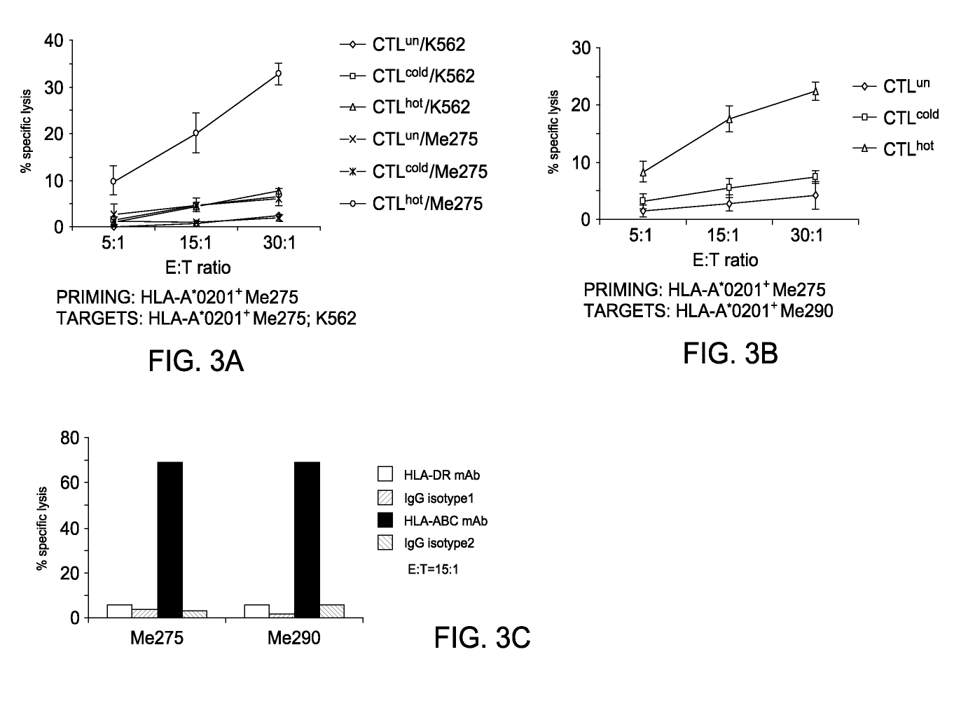

DCs Loaded with Hot Melanoma Bodies Rapidly Yield CTLs Able To Kill Melanoma Cells In 4-Hour ⁵¹Cr Release Assay. It has been previously reported that naïve CD8⁺ T cells require three stimulations with cold melanoma body-loaded DCs to differentiate into melanoma-specific CTLs (Berard et al., 2000). Therefore, to assess whether loading with hot melanoma bodies enhances the immunogenicity of loaded DCs, CTL differentiation after two rounds of stimulation was measured. As shown in FIG. 3A-3C, HLA-A*0201⁺CD8⁺ T cells stimulated twice with hot melanoma body-loaded DCs were able to kill HLA-A*0201+ Me275 melanoma cells used as a source of melanoma bodies with 33%±3 specific lysis at the E:T ratio 30:1 (n=3, FIG. 3A). The killing was specific as no lysis of K562 cells was found. As expected, CD8⁺ T cells stimulated twice with cold melanoma body-loaded DCs were not able to kill melanoma cells (FIG. 3A). Furthermore, after two stimulations, the CD8⁺ T cells primed with hot Me275 melanoma body-loaded DCs were able to kill HLA-A*0201+ Me290 melanoma cells (n=3, FIG. 3B), suggesting priming against antigens shared between these two melanoma cell lines. Killing of melanoma cells was restricted by their expression of MHC class I, as the pretreatment of target cells with MHC class I blocking mAb W6/32 resulted in >60% inhibition of Me275 and Me290 killing at different E:T ratios (15% lysis at E:T ratio 15:1 without W6/32 mAb and 4% lysis with W6/32 mAb, FIG. 3C and data not shown).

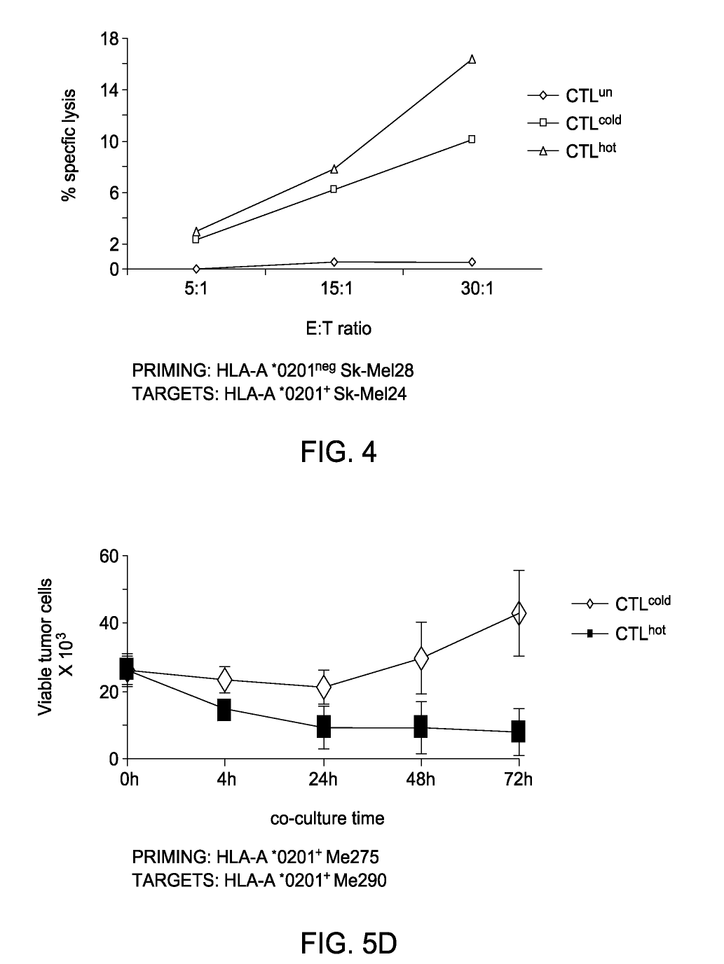

Furthermore, HLA-A*0201⁺ DCs loaded with hot melanoma bodies derived from HLA-A*0201^{neg} Sk-Mel28 melanoma cells elicited CD8⁺ T cells able to kill, albeit at lower efficiency, HLA-A*0201⁺ Sk-Mel24 melanoma cells (16% of specific lysis at E:T ratio 30:1, FIG. 4). These results indicate cross-priming against shared melanoma antigens. Thus, loading DCs with hot melanoma bodies enhances their immunogenicity as two stimulations are sufficient to induce naïve CD8⁺ T cell differentiation into CTLs able to kill melanoma cell lines.

### EXAMPLE 14

DCs Loaded with Hot Melanoma Bodies Rapidly Yield CTLs Able To Control the Survival/Growth of Melanoma Cells. The present inventors have shown that the Tumor Regression Assay (TRA) allows detection of T cell-dependent inhibition of tumor survival/growth that might serve as a measure of T cell capacity to prevent relapse (Paczesny et al., 2004). Therefore, TRA was used as another measure of the enhanced immunogenicity of hot melanoma body-loaded DCs. To this end, CD8⁺ T cells from cultures with either cold or hot melanoma body-loaded DCs were co-cultured with EGFP-labeled melanoma cells (at the E:T ratio 20:1); and the cultures were harvested at different time points, labeled with anti-CD8-PE and analyzed by flow cytometry.

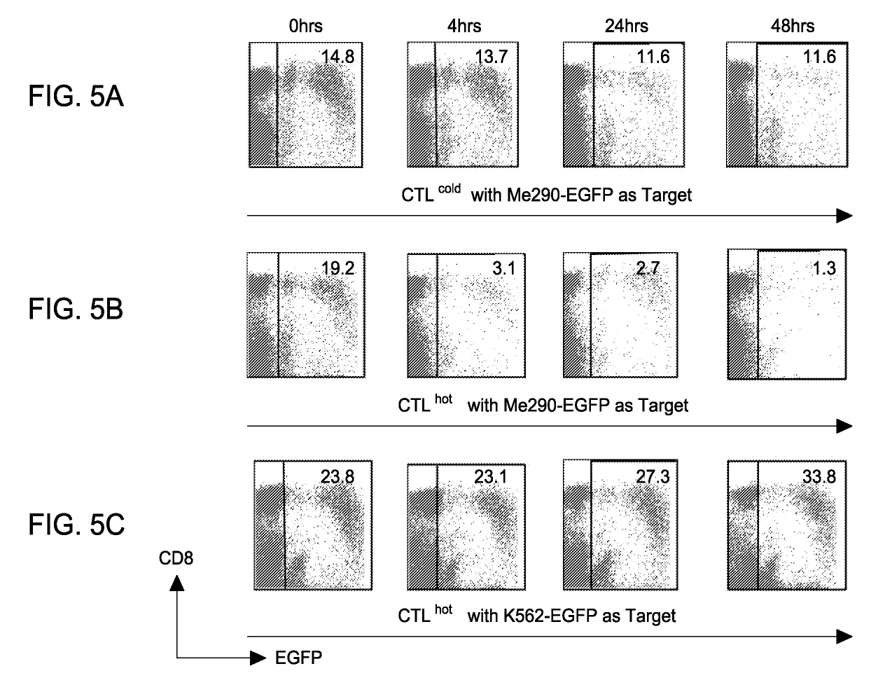

FIGS. 5A-5D show a representative study where HLA-A*0201⁺ T cells primed, in two-week cultures, against HLA-A*0201⁺ Me290 melanoma cells were tested for their capacity to inhibit the survival/growth of Me290 melanoma and control K562 cells. As expected, CD8⁺ T cells primed with cold Me290 bodies were not very efficient in the control of Me290 growth. Indeed, after 4 hours of co-culture, the fraction of EGFP⁺ (viable) melanoma cells was nearly identical as compared to the onset of co-culture (FIG. 5A). After 24 hours, approximately 20% decrease in the fraction of viable melanoma cells was observed (FIG. 5A). On the contrary, CD8⁺ T cells primed with hot melanoma bodies loaded DCs were very efficient in controlling Me290 cell survival/growth (FIG. 5B); after 4 hours of culture, the fraction of EGFP⁺ melanoma cells was >80% decreased and remained low over 48 hours of co-culture. The observed decrease in the fraction of viable tumor cells was specific to melanoma, as the survival/growth of NK-sensitive K562 cells was not altered (FIG. 5C).

The priming of CD8⁺ T cells specific against melanoma cell lines was further confirmed by the ability of HLA-A*0201⁺CD8⁺ T cells primed against HLA-A*0201⁺ Me275 melanoma cells to inhibit the growth of HLA-A*0201⁺ Me290 melanoma cells (FIG. 5D). Here, the survival/growth of melanoma cells was measured by Trypan blue exclusion and viable cell count using light microscopy. In three studies, CD8⁺ T cells primed with hot Me275 melanoma body-loaded DCs were considerably more efficient than those primed with cold Me275 body-loaded DCs in control of the growth/survival of Me290 melanoma cells (FIG. 5D). Thus, loading DCs with hot melanoma bodies enhances their immunogenicity as only two stimulations are necessary to induce naïve CD8⁺ T cell differentiation into CTLs able to control the growth/survival of melanoma cell lines.

### EXAMPLE 15

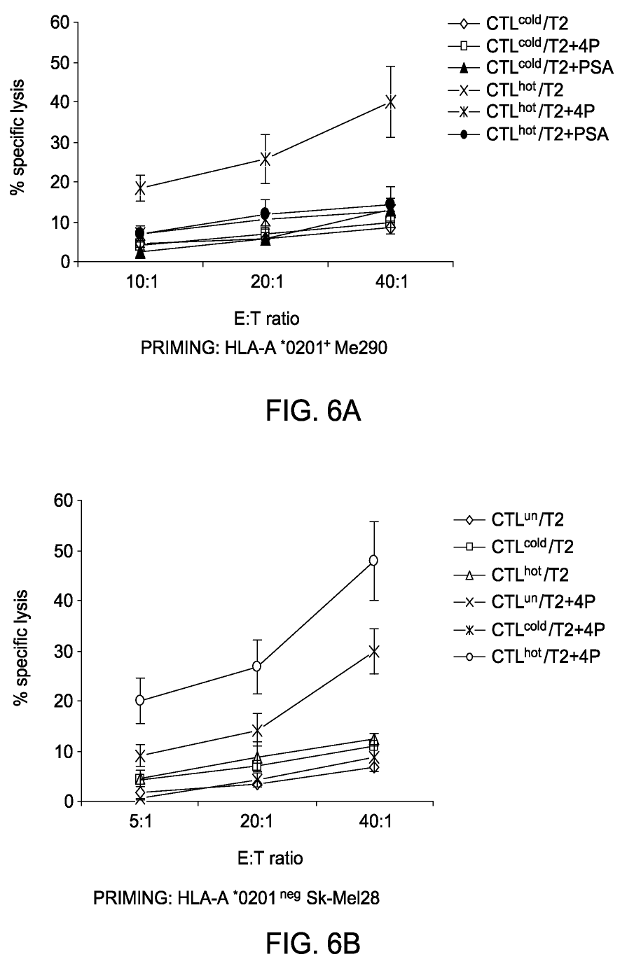

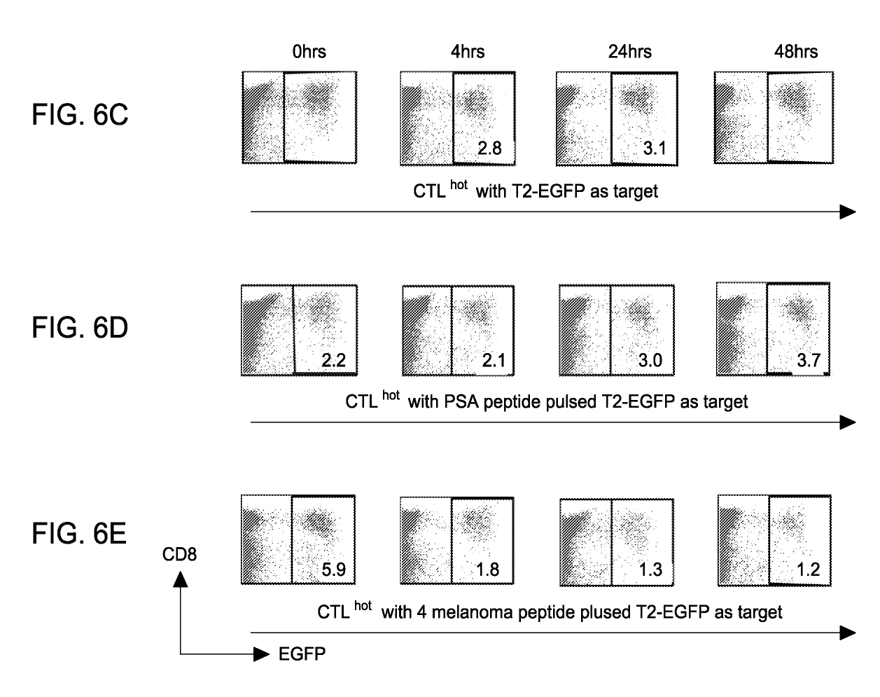

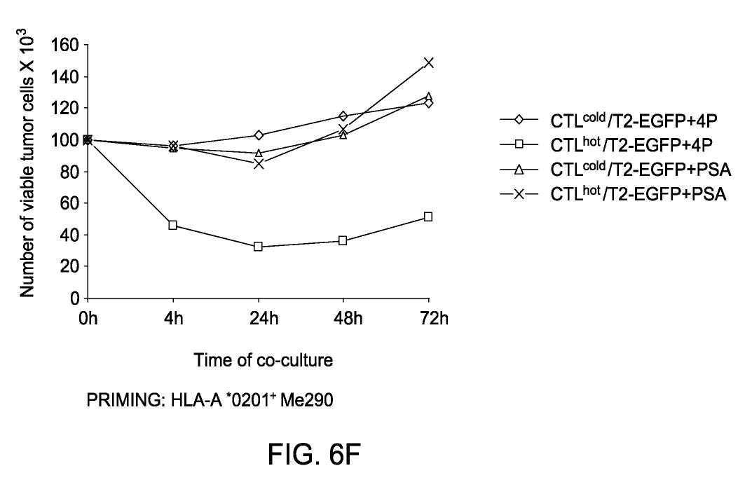

DCs Loaded with Hot Melanoma Bodies Rapidly Yield CTLs Able To Recognize Melanoma Differentiation Antigen-Derived Peptides. It was further determined whether the CD8⁺ T cells primed with hot body-loaded DCs are specific for the melanoma differentiation antigens: MART-1/Melan A, gp100, tyrosinase and MAGE-3. T cell specificity was assessed by their capacity to recognize melanoma peptides presented on T2 cells in two assays: 1) ⁵¹Cr release after 4 hours of co-culture with ⁵¹Cr labeled T2 cells pulsed with a mix of the four melanoma peptides derived from differentiation antigens, and 2) survival of melanoma peptide-pulsed EGFP-expressing T2 cells in the TRA. It was found that CD8⁺ T cells primed with hot HLA-A*0201⁺ Me290 melanoma bodies can kill melanoma-peptide pulsed T2 cells with 40% specific lysis in the ⁵¹Cr release assay at the E:T ratio 40:1 (FIG. 6A). The killing was specific as T2 cells pulsed with a control PSA peptide were not killed. As expected, CD8⁺ T cells primed with cold Me290 melanoma body-loaded DCs were unable to kill peptide-pulsed T2 cells (FIG. 6A), consistent with our earlier observations that three stimulations are necessary (Berard et al., 2000). Induction of melanoma differentiation antigen-specific T cells was further confirmed in the cross-priming study. HLA-A*0201⁺CD8⁺ T cells were stimulated twice with DCs loaded with hot melanoma bodies derived from HLA-A*0201^{neg} Sk-Mel28 cells. As shown in FIG. 6B, primed CD8⁺ T cells killed melanoma peptide-pulsed T2 cells with 48%±8 specific lysis (E:T ratio 40:1, n=3), but not PSA peptide-pulsed T2 cells, thus indicating cross-priming.

The capacity of primed CD8⁺ T cells to recognize melanoma antigens was also confirmed in a tumor regression assay. CD8⁺ T cells from two-week cultures with loaded DCs were co-cultured with EGFP-expressing T2 cells either unpulsed, pulsed with control PSA peptide or pulsed with a mix of the four melanoma peptides. The survival of T2 cells was measured at different time points as the fraction of EGFP⁺ cells in flow cytometry. As shown in FIG. 6E, primed CD8⁺ T cells induced a considerable (approximately 70%) decrease in the fraction of EGFP⁺ melanoma peptide-pulsed T2 cells already after a 4-hour co-culture, and the fraction of EGFP⁺ T2 cells remained low over 48 hours of co-culture. This effect was specific as the survival of control T2 cells (either unpulsed or PSA-pulsed) was not altered (FIGS. 6C and 6D, respectively). According to the FACS data, the growth rates of peptide pulsed T2-EGFP cells were calculated by using the following formula:

$$\% growth rate = \frac{\% EGFP + population at given time point}{\% EGFP + population at 0 hour} \times 100 \%$$

wherein the tumor growth rate at 0 hrs was defined as 100%. As shown in FIG. 6F, CD8⁺ T cells primed with hot melanoma body-loaded DCs were much more efficient than those primed with cold melanoma body-loaded DCs, whereas CD8⁺ T cells primed with cold melanoma body-loaded DCs were unable to control the survival/growth of melanoma-peptide pulsed T2 cells in three independent studies. Thus, loading DCs with hot melanoma bodies enhanced their immunogenicity and two stimulations were sufficient to induce naïve CD8⁺ T cell differentiation into melanoma-specific CTLs.

### EXAMPLE 16

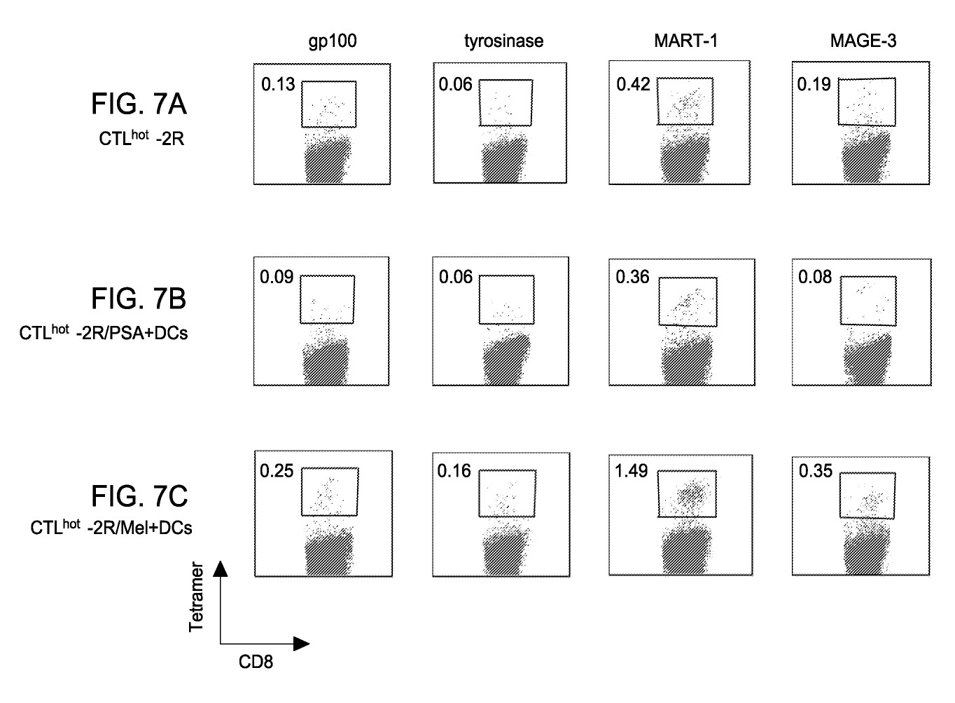

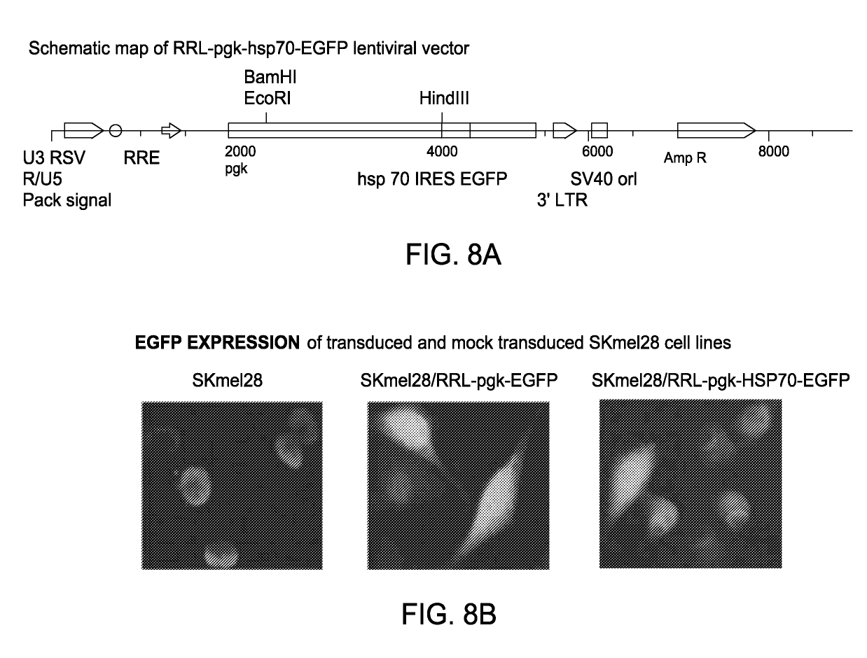

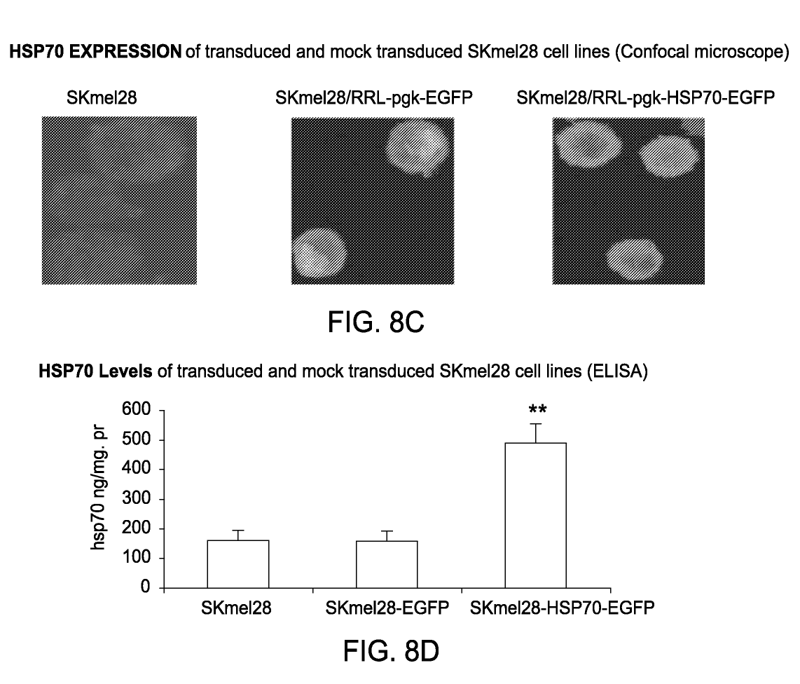

DCs Loaded with Hot Melanoma Bodies Promptly Yield Melanoma Tetramer Binding CD8⁺ T Cells. The frequency of melanoma-specific CD8⁺ T cells was measured using tetramers loaded with the four melanoma peptides i.e., gp100, MART-1/Melan A, tyrosinase and MAGE-3. FIGS. 7A-7C show a representative pattern of tetramer staining. After two stimulations with hot HLA-A*0201⁺ Me290 melanoma body-loaded DCs, 0.4% of CD8⁺ T cells were specific for MART-1/Melan A (FIG. 7A). However, other specificities could barely be detected upon acquisition of 5×10⁴ T cells for analysis, indicating that either the T cells were primed only against MART-1 or that the elicited repertoire was broad but at the low frequency for a given peptide and, therefore, made it difficult to detect a T cell with particular specificity.

To address this, the presence of recall memory CD8⁺ T cells was analyzed, i.e., T cells that require a single restimulation with defined peptide-pulsed DCs for expansion. Naïve CD8⁺ T cells were primed in 2-week cultures with DCs loaded with hot Me290 melanoma bodies as described above. At Day 7 after the second stimulation, the T cells were washed, restimulated with autologous DCs pulsed either with each of the four melanoma peptides or with a control PSA peptide, and analyzed after an additional 7 days of culture. As shown in FIG. 7B, the frequency of melanoma tetramer binding CD8⁺ T cells remained stable after restimulation with PSA-peptide pulsed DCs. However, a boost with melanoma peptide-pulsed DCs resulted in the expansion of melanoma-specific CD8⁺ T cells (FIG. 7C). Thus, the frequency of MART-1/Melan A tetramer binding CD8⁺ T cells increased to 1.49%, and the T cells with other specificities were clearly detectable: 0.35% MAGE-3 specific CD8⁺ T cells, 0.25% gp100 specific CD8⁺ T cells, and 0.16% tyrosinase specific CD8⁺ T cells.

Similar results were obtained in two studies with CD8⁺ T cells primed against hot HLA-A*0201⁺ Me290 melanoma cells as well as in the cross-priming situation where the HLA-A*0201⁺ T cells were primed against HLA-A*0201^{neg} Sk-Mel28 melanoma cells (Table I). In the latter case, the recall memory T cells primed by melanoma body-loaded DCs and expanded by boost with melanoma peptide-pulsed DCs were clearly detectable with predominant specificity for MART-1/Melan A, tyrosinase and MAGE-3 (Table 1). Finally, in both cases, i.e., whether the T cells were primed with HLA-A*0201⁺ Me290 melanoma bodies or HLA-A*0201^{neg} Sk-Mel28 melanoma bodies, DCs loaded with hot bodies were far more efficient in priming melanoma specific CD8⁺ T cells than DCs loaded with cold bodies (Table I).

TABLE I
Frequency of Melanoma Tetramer Binding T cells

| | | Gp100 | Tyrosinase | MART-1 | MAGE-3 |
|---|---|---|---|---|---|
| Cold Me290 | 2st | 0.02 | 0.08 | 0.2 | 0.04 |
| | 2st + | 0.02 | 0.02 | 0.13 | 0.05 |
| | PSA-DCs | | | | |
| | 2st + | 0.03 | 0.11 | 0.67 | 0.11 |
| | Mel-DCs | | | | |
| Hot Me290 | 2st | 0.13 | 0.06 | 0.42 | 0.19 |
| | 2st + | 0.09 | 0.06 | 0.36 | 0.08 |
| | PSA-DCs | | | | |
| | 2st + | 0.25 | 0.16 | 1.49 | 0.35 |
| | Mel-DCs | | | | |
| Cold Sk-Mel28 | 2st | 0.01 | 0.01 | 0.04 | 0.07 |
| | 2st + | 0.01 | 0.04 | 0.08 | 0.07 |
| | PSA-DCs | | | | |
| | 2st + | 0.05 | 0.04 | 0.1 | 0.04 |
| | Mel-DCs | | | | |
| Hot Sk-Mel28 | 2st | 0.02 | 0.07 | 0.12 | 0.05 |
| | 2st + | 0.01 | 0.03 | 0.16 | 0.09 |
| | PSA-DCs | | | | |
| | 2st + | 0.02 | 0.14 | 0.36 | 0.15 |
| | Mel-DCs | | | | |

### EXAMPLE 17

Heat treatment Increases Cross-Priming via Up-regulation of Transcription of Genes Encoding Tumor Antigens. The effect of heat treatment on the transcription of genes encoding tumor antigens was investigated using real-time reverse transcriptase-polymerase chain reaction (RT-PCR) analysis.

For a given cell line of interest, total RNA was extracted using the RNeasy kit (Qiagen, Valencia, Calif.) according to manufacturer's instructions and assessed using an Agilent 2100 Bioanalyzer (Agilent Palo Alto, Calif.). RNAs were subjected to a second DNase treatment with the TURBO DNA-free kit (Ambion, Inc., Austin, Tex.). From 100 nanograms of RNA, cDNA was synthesized using the Two-Cycle cDNA Synthesis kit (Affymetrix, Inc., Santa Clara, Calif.) followed by in vitro transcription (MEGAscript T7 kit, Ambion, Inc.). Two-step RT-PCR was performed using Applied Biosystems TaqMan Assays on Demand probe and primer sets according to the manufacturers' instructions (Applied Biosystems, Inc. Foster City, Calif.). Reverse transcription was carried out using the High Capacity cDNA Archive Kit (Applied Biosystems). Real-time PCR was performed on an ABI Prism 7700 Sequence Detection System. Relative mRNA expression was calculated using the comparative Cₜ method as described in Affymetrix User Bulletin #2 (updated October, 2001). Results were calculated as the normalized difference in Cₜ for heat treated melanoma cells relative to untreated melanoma cells (ΔΔC_{t,q}).

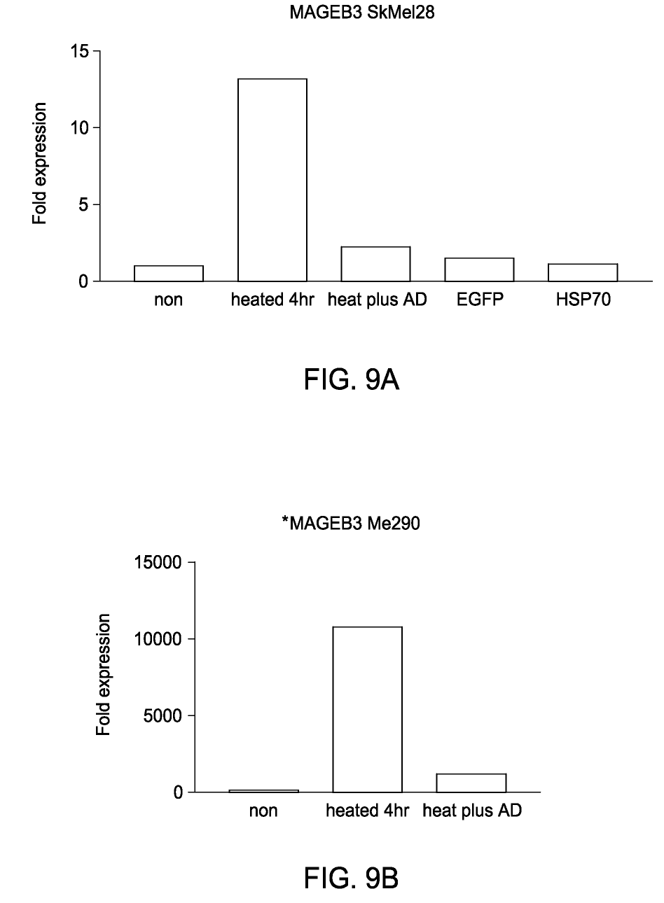

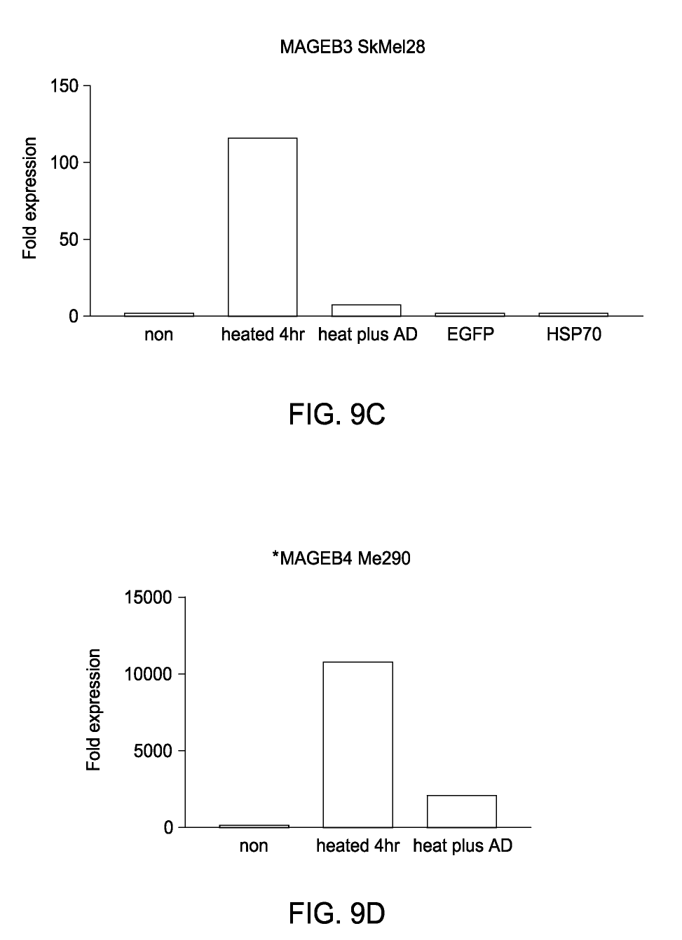

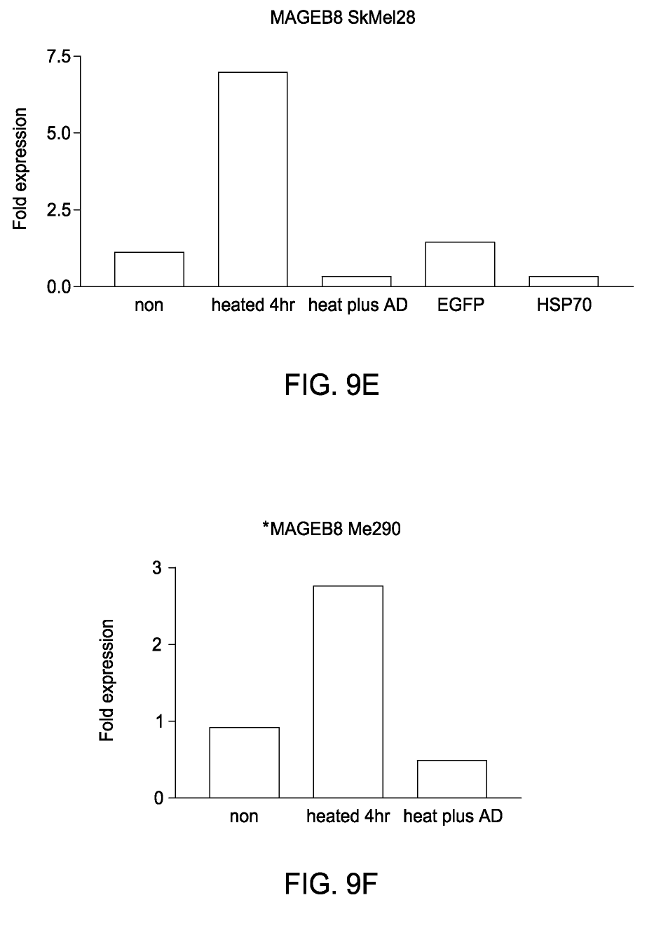

The results of the study are given in FIG. 9A-9F. SkMel28 cells were either not heated (“non”); heat treated at 42 degrees C. for 4 hours (“heated 4 hr”); exposed to Actinomycin D, a known transcription inhibitor, during heat treatment at 42 degrees C. for 4 hours (“heat plus AD”); transfected with a control vector EGFP (“EGFP”); or transfected with a vector expression HSP70 (“HSP70”) prior to real-time RT-PCR analysis of the mRNA expression of MAGE-B3 (FIG. 9A), MAGE-B4 (FIG. 9C) or MAGE-A8 (FIG. 9E). Comparing the non-heated cells to the heat-treated cells, a definite up-regulation of the transcription of the tumor antigens MAGE-B3, MAGE-B4, and MAGE-A8 was observed after heat treatment. The addition of Actinomycin D inhibited the upregulation, confirming transcriptional regulation. Over-expression of HSP70 did not result in increased transcription of the melanoma antigens. Similar results were obtained in the Me290 cell line. Cells were either not heated (“non”); heat treated at 42 degrees C. for 4 hours (“heated 4 hr”); exposed to Actinomycin D, a known transcription inhibitor, during heat treatment at 42 degrees C. for 4 hours (“heat plus AD”) prior to real-time RT-PCR analysis of the mRNA expression of MAGE-B3 (FIG. 9B), MAGE-B4 (FIG. 9D) or MAGE-A8 (FIG. 9F). Comparing the non-heated cells to the heat-treated cells, a definite up-regulation of the transcription of the tumor antigens MAGE-B3, MAGE-B4, and MAGE-A8 was observed after heat treatment. The addition of Actinomycin D inhibited the upregulation, confirming transcriptional regulation. Thus, heat treatment, or hyperthermia, increased cross-priming via up-regulation of the transcription of genes encoding the melanoma antigens.

In summary, heat treatment of melanoma cells prior to induction of melanoma cell death and generation of melanoma bodies to be loaded onto DC vaccine according to the methods of the present invention results in the considerable enhancement of the immunogenicity of such DC vaccine. The enhanced immunogenicity and, thus, patient response to such therapy can easily be detected in several assays measuring priming of naïve CD8⁺ T cells: i) the number of stimulations with loaded DCs needed for naïve CD8⁺ T cell differentiation, ii) the killing of HLA-A*0201 melanoma cells in a standard 4 hour ⁵¹Cr release assay, iii) the capacity to prevent tumor growth in vitro in a tumor regression assay, iv) killing of melanoma peptide-pulsed T2 cells, and v) binding of melanoma tetramers. In all assays, DCs loaded with heated melanoma bodies are superior to DCs loaded with unheated, or cold, melanoma bodies. These results suggest that not only the quantity but also the quality of primed T cells is superior when heated melanoma cells are used as a source of melanoma antigens to be loaded onto DC vaccine. Clinical applications of the methods of the present invention on DC based or T cell based tumor immunotherapy include use of the increased immunogenicity of the DC vaccines of the present invention to 1) shorten the time necessary for T cell elicitation/expansion for adoptive T cell therapy protocols and 2) limit the number of DCs per injection and/or the times of DC injections in DC-based immunotherapy protocols.

Examples of additional human tumor cell lines that may be used with the present invention include, for example:

TABLE II
Cancer Cells

| CELL LINE | TUMOR TYPE |
|---|---|
| J82 | Transitional-cell carcinoma, bladder |
| RT4 | Transitional-cell papilloma, bladder |
| ScaBER | Squamous carcinoma, bladder |
| T24 | Transitional-cell carcinoma, bladder |
| TCCSUP | Transitional-cell carcinoma, bladder, primary grade IV |
| 5637 | Carcinoma, bladder, primary |
| SK-N-MC | Neuroblastoma, metastasis to supra-orbital area |
| SK-N-SH | Neuroblastoma, metastasis to bone marrow |
| SW 1088 | Astrocytoma |
| SW 1783 | Astrocytoma |
| U-87 MG | Glioblastoma, astrocytoma, grade III |
| U-118 MG | Glioblastoma |
| U-138 MG | Glioblastoma |
| U-373 MG | Glioblastoma, astrocytoma, grade III |
| Y79 | Retinoblastoma |
| BT-20 | Carcinoma, breast |
| BT-474 | Ductal carcinoma, breast |
| MCF7 | Breast adenocarcinoma, pleural effusion |
| MDA-MB-134-V | Breast, ductal carcinoma, pleural I effusion |
| MDA-MD-157 | Breast medulla, carcinoma, pleural effusion |
| MDA-MB-175-VII | Breast, ductal carcinoma, pleural |
| | effusion |
| MDA-MB-361 | Adenocarcinoma, breast, metastasis to brain |
| SK-BR-3 | Adenocarcinoma, breast, malignant pleural effusion |
| C-33 A | Carcinoma, cervix |
| HT-3 | Carcinoma, cervix, metastasis to lymph node |
| ME-180 | Epidermoid carcinoma, cervix, metastasis to omentum |
| MEL-175 | Melanoma |
| MEL-290 | Melanoma |
| HLA-A*0201 | Melanoma cells |
| MS751 | Epidermoid carcinoma, cervix, metastasis to lymph node |
| SiHa | Squamous carcinoma, cervix |
| JEG-3 | Choriocarcinoma |
| Caco-2 | Adenocarcinoma, colon |
| HT-29 | Adenocarcinoma, colon, moderately well-differentiated grade |
| | II |
| SK-CO-1 | Adenocarcinoma, colon, ascites |
| HuTu 80 | Adenocarcinoma, duodenum |
| A-253 | Epidermoid carcinoma, submaxillary gland |
| FaDu | Squamous cell carcinoma, pharynx |
| A-498 | Carcinoma, kidney |
| A-704 | Adenocarcinoma, kidney |
| Caki-1 | Clear cell carcinoma, consistent with renal primary, |
| | metastasis to skin |
| Caki-2 | Clear cell carcinoma, consistent with renal primary |
| SK-NEP-1 | Wilms' tumor, pleural effusion |
| SW 839 | Adenocarcinoma, kidney |
| SK-HEP-1 | Adenocarcinoma, liver, ascites |
| A-427 | Carcinoma, lung |
| Calu-1 | Epidermoid carcinoma grade III, lung, metastasis to pleura |
| Calu-3 | Adenocarcinoma, lung, pleural effusion |
| Calu-6 | Anaplastic carcinoma, probably lung |
| SK-LU-1 | Adenocarcinoma, lung consistent with poorly differentiated, |
| | grade III |
| SK-MES-1 | Squamous carcinoma, lung, pleural effusion |
| SW 900 | Squamous cell carcinoma, lung |
| EB1 | Burkitt lymphoma, upper maxilia |
| EB2 | Burkitt lymphoma, ovary |
| P3HR-1 | Burkitt lymphoma, ascites |
| HT-144 | Malignant melanoma, metastasis to subcutaneous tissue |
| Malme-3M | Malignant melanoma, metastasis to lung |
| RPMI-7951 | Malignant melanoma, metastasis to lymph node |
| SK-MEL-1 | Malignant melanoma, metastasis to lymphatic system |
| SK-MEL-2 | Malignant melanoma, metastasis to skin of thigh |
| SK-MEL-3 | Malignant melanoma, metastasis to lymph node |
| SK-MEL-5 | Malignant melanoma, metastasis to axillary node |
| SK-MEL-24 | Malignant melanoma, metastasis to node |
| SK-MEL-28 | Malignant melanoma |
| SK-MEL-31 | Malignant melanoma |
| Caov-3 | Adenocarcinoma, ovary, consistent with primary |
| Caov-4 | Adenocarcinoma, ovary, metastasis to subserosa of fallopian |
| | tube |
| SK-OV-3 | Adenocarcinoma, ovary, malignant ascites |
| SW 626 | Adenocarcinoma, ovary |
| Capan-1 | Adenocarcinoma, pancreas, metastasis to liver |
| Capan-2 | Adenocarcinoma, pancreas |
| DU 145 | Carcinoma, prostate, metastasis to brain |
| A-204 | Rhabdomyosarcoma |
| Saos-2 | Osteogenic sarcoma, primary |
| SK-ES-1 | Anaplastic osteosarcoma versus Swing sarcoma, bone |
| SK-LNS-1 | Leiomyosarcoma, vulva, primary |
| SW 684 | Fibrosarcoma |
| SW 872 | Liposarcoma |
| SW 982 | Axilla synovial sarcoma |
| SW 1353 | Chondrosarcoma, humerus |
| U-2 OS | Osteogenic sarcoma, bone primary |
| Malme-3 | Skin fibroblast |
| KATO III | Gastric carcinoma |
| Cate-1B | Embryonal carcinoma, testis, metastasis to lymph node |
| Tera-1 | Embryonal carcinoma, malignancy consistent with metastasis to lung |
| Tera-2 | Embryonal carcinoma, malignancy consistent with, metastasis |
| | to lung |
| SW579 | Thyroid carcinoma |
| AN3 CA | Endometrial adenocarcinoma, metastatic |
| HEC-1-A | Endometrial adenocarcinoma |
| HEC-1-B | Endometrial adenocarcinoma |
| SK-UT-1 | Uterine, mixed mesodermal tumor, consistent with |
| | leiomyosarcoma grade III |
| SK-UT-1B | Uterine, mixed mesodermal tumor, consistent with |
| | leiomyosarcoma grade III |
| Sk-Mel28 | Melanoma |
| SW 954 | Squamous cell carcinoma, vulva |
| SW 962 | Carcinoma, vulva, lymph node metastasis |
| NCI-H69 | Small cell carcinoma, lung |
| NCI-H128 | Small cell carcinoma, lung |
| BT-483 | Ductal carcinoma, breast |
| BT-549 | Ductal carcinoma, breast |
| DU4475 | Metastatic cutaneous nodule, breast carcinoma |
| HBL-100 | Breast |
| Hs 578Bst | Breast, normal |
| Hs 578T | Ductal carcinoma, breast |
| MDA-MB-330 | Carcinoma, breast |
| MDA-MB-415 | Adenocarcinoma, breast |
| MDA-MB-435S | Ductal carcinoma, breast |
| MDA-MB-436 | Adenocarcinoma, breast |
| MDA-MB-453 | Carcinoma, breast |
| MDA-MB-468 | Adenocarcinoma, breast |
| T-47D | Ductal carcinoma, breast, pleural effusion |
| Hs 766T | Carcinoma, pancreas, metastatic to lymph node |
| Hs 746T | Carcinoma, stomach, metastatic to left leg |
| Hs 695T | Amelanotic melanoma, metastatic to lymph node |
| Hs 683 | Glioma |
| Hs 294T | Melanoma, metastatic to lymph node |
| Hs 602 | Lymphoma, cervical |
| JAR | Choriocarcinoma, placenta |
| Hs 445 | Lymphoid, Hodgkin's disease |
| Hs 700T | Adenocarcinoma, metastatic to pelvis |
| H4 | Neuroglioma, brain |
| Hs 696 | Adenocarcinoma primary, unknown, metastatic to bone-sacrum |
| Hs 913T | Fibrosarcoma, metastatic to lung |
| Hs 729 | Rhabdomyosarcoma, left leg |
| FHs 738Lu | Lung, normal fetus |
| FHs 173We | Whole embryo, normal |
| FHs 738Bl | Bladder, normal fetus |
| NIH:OVCAR-3 | Ovary, adenocarcinoma |
| Hs 67 | Thymus, normal |
| RD-ES | Ewing's sarcoma |
| ChaGo K-1 | Bronchogenic carcinoma, subcutaneous metastasis, human |
| WERI-Rb-1 | Retinoblastoma |
| NCI-H446 | Small cell carcinoma, lung |
| NCI-H209 | Small cell carcinoma, lung |
| NCI-H146 | Small cell carcinoma, lung |
| NCI-H441 | Papillary adenocarcinoma, lung |
| NCI-H82 | Small cell carcinoma, lung |
| H9 | T-cell lymphoma |
| NCI-H460 | Large cell carcinoma, lung |
| NCI-H596 | Adenosquamous carcinoma, lung |
| NCI-H676B | Adenocarcinoma, lung |
| NCI-H345 | Small cell carcinoma, lung |
| NCI-H820 | Papillary adenocarcinoma, lung |
| NCI-H520 | Squamous cell carcinoma, lung |
| NCI-H661 | Large cell carcinoma, lung |
| NCI-H510A | Small cell carcinoma, extra-pulmonary origin, metastatic |
| D283 Med | Medulloblastoma |
| Daoy | Medulloblastoma |
| D341 Med | Medulloblastoiua |
| AML-193 | Acute monocyte leukemia |
| MV4-11 | Leukemia biphenotype |

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

Albert, M. L., B. Sauter, and N. Bhardwaj. 1998a. Dendritic cells acquire antigen from apoptotic cells and induce class I-restricted CTLs. Nature 392:86.
Albert, M. L., J. C. Darnell, A. Bender, L. M. Francisco, N. Bhardwaj, and R. B. Darnell. 1998b. Tumor-specific killer cells in paraneoplastic cerebellar degeneration. Nat Med 4:1321.
Asea, A., M. Rehli, E. Kabingu, J. A. Boch, O. Bare, P. E. Auron, M. A. Stevenson, and S. K. Calderwood. 2002. Novel signal transduction pathway used by extracellular HSP70: role of toll-like receptor (TLR) 2 and TLR4. J Biol Chem 277:15028.
Ashley, D. M., B. Faiola, S, Nair, L. P. Hale, D. D. Bigner, and E. Gilboa. 1997. Bone marrow-generated dendritic cells pulsed with tumor extracts or tumor RNA induce antitumor immunity against central nervous system tumors. J Exp Med 186:1177.
Banchereau, J., A. K. Palucka, M. Dhodapkar, S. Burkeholder, N. Taquet, A. Rolland, S. Taquet, S. Coquery, K. M. Wittkowski, N. Bhardwaj, L. Pineiro, R. Steinman, and J. Fay. 2001. Immune and clinical responses in patients with metastatic melanoma to CD34(+) progenitor-derived dendritic cell vaccine. Cancer Res 61:6451.
Basu, S., and P. K. Srivastava. 2000. Heat shock proteins: the fountainhead of innate and adaptive immune responses. Cell Stress Chaperones 5:443.
Basu, S., R. J. Binder, T. Ramalingam, and P. K. Srivastava. 2001. CD91 is a common receptor for heat shock proteins gp96, hsp90, hsp70, and calreticulin. Immunity 14:303.
Becker, T., F. U. Hartl, and F. Wieland. 2002. CD40, an extracellular receptor for binding and uptake of Hsp70-peptide complexes. J Cell Biol 158:1277.
Berard, F., P. Blanco, J. Davoust, E. M. Neidhart-Berard, M. Nouri-Shirazi, N. Taquet, D. Rimoldi, J. C. Cerottini, J. Banchereau, and A. K. Palucka. 2000. Cross-priming of naive CD8 T cells against melanoma antigens using dendritic cells loaded with killed allogeneic melanoma cells. J Exp Med 192:1535.
Boczkowski, D., S. K. Nair, D. Snyder, and E. Gilboa. 1996. Dendritic cells pulsed with RNA are potent antigen-presenting cells in vitro and in vivo. J Exp Med 184:465.
Castelli, C., A. M. Ciupitu, F. Rini, L. Rivoltini, A. Mazzocchi, R. Kiessling, and G. Parmiani. 2001. Human heat shock protein 70 peptide complexes specifically activate antimelanoma T cells. Can Res 61:222-227.
Delneste, Y., G. Magistrelli, J. Gauchat, J. Haeuw, J. Aubry, K. Nakamura, N. Kawakami-Honda, L. Goetsch, T. Sawamura, J. Bonnefoy, and P. Jeannin. 2002. Involvement of LOX-1 in dendritic cell-mediated antigen cross-presentation. 1 mm unity 17:353.
Dhodapkar, M. V., R. M. Steinman, M. Sapp, H. Desai, C. Fossella, J. Krasovsky, S. M. Donahoe, P. R. Dunbar, V. Cerundolo, D. F. Nixon, and N. Bhardwaj. 1999. Rapid generation of broad T-cell immunity in humans after a single injection of mature dendritic cells. J Clin Invest 104:173.
Dhodapkar, M. V., J. Krasovsky, R. M. Steinman, and N. Bhardwaj. 2000. Mature dendritic cells boost functionally superior CD8(+) T-cell in humans without foreign helper epitopes [see comments]. J Clin Invest 105:R9.
Fong, L., and E. G. Engleman. 2000. Dendritic cells in cancer immunotherapy. Annu Rev Immunol 18:245.
Frydman, J. 2001. Folding of newly translated proteins in vivo: the role of molecular chaperones. Annu Rev Biochem 70:603.
Gilboa, E. 1999. The makings of a tumor rejection antigen. Immunity 11:263.
Heiser, A., D. Coleman, J. Dannull, D. Yancey, M. A. Maurice, C. D. Lallas, P. Dahm, D. Niedzwiecki, E. Gilboa, and J. Vieweg. 2002. Autologous dendritic cells transfected with prostate-specific antigen RNA stimulate CTL responses against metastatic prostate tumors. J Clin Invest 109:409.
Kierstead, L. S., E. Ranieri, W. Olson, V. Brusic, J. Sidney, A. Sette, Y. L. Kasamon, C. L. Slingluff, Jr., J. M. Kirkwood, and W. J. Storkus. 2001. gp100/pmel17 and tyrosinase encode multiple epitopes recognized by Th1-type CD4+ T cells. Br J Cancer 85:1738.
Labarriere, N., L. Bretaudeau, N. Gervois, M. Bodinier, G. Bougras, E. Diez, F. Lang, M. Gregoire, and F. Jotereau. 2002. Apoptotic body-loaded dendritic cells efficiently cross-prime cytotoxic T lymphocytes specific for NA17-A antigen but not for Melan-A/MART-1 antigen. Int J Cancer 101:280.
Nestle, F. O., S. Alijagic, M. Gilliet, Y. Sun, S. Grabbe, R. Dummer, G. Burg, and D. Schadendorf. 1998. Vaccination of melanoma patients with peptide- or tumor lysate-pulsed dendritic cells. Nat Med 4:328.
Noessner, E, R. Gastpar, V. Milani, A. Brandl, P. J. Hutzler, M. C. Kuppner, M. Roos, E. Kremmer, A. Asea, S. K. Calderwood, and R. D. Issels. 2002. Tumor-derived heat shock protein 70 peptide complexes are cross-presented by human dendritic cells. J Immunol 169:5424-5432.
Nouri-Shirazi, M., J. Banchereau, D. Bell, S. Burkeholder, E. T. Kraus, J. Davoust, and K. A. Palucka. 2000. Dendritic cells capture killed tumor cells and present their antigens to elicit tumor-specific immune responses. J Immunol 165:3797.
Paczesny, S., J. Banchereau, K. M. Wittkowski, G. Saracino, J. Fay, and A. K. Palucka. 2004. Expansion of Melanoma-specific Cytolytic CD8+ T Cell Precursors in Patients with Metastatic Melanoma Vaccinated with CD34+ Progenitor-derived Dendritic Cells. J Exp Med 199:1503.
Palucka, A. K., M. V. Dhodapkar, S. Paczesny, S. Burkeholder, K. M. Wittkowski, R. M. Steinman, J. Fay, and J. Banchereau. 2003. Single injection of CD34+ progenitor-derived dendritic cell vaccine can lead to induction of T-cell immunity in patients with stage 1V melanoma. J Immunother 26:432.
Regnault, A., D. Lankar, V. Lacabanne, A. Rodriguez, C. Thery, M. Rescigno, T. Saito, S. Verbeek, C. Bonnerot, P. Ricciardi-Castagnoli, and S. Amigorena. 1999. Fcgamma receptor-mediated induction of dendritic cell maturation and major histocompatibility complex class I-restricted antigen presentation after immune complex internalization. J Exp Med 189:371.
Ribas, A., L. H. Butterfield, J. A. Glaspy, and J. S. Economou. 2002. Cancer immunotherapy using gene-modified dendritic cells. Curr Gene Ther 2:57.
Srivastava, P. K., H. Udono, N. E. Blachere, and Z. Li. 1994. Heat shock proteins transfer peptides during antigen processing and CTL priming. Immunogenetics 39:93.
Srivastava, P. 2002. Interaction Of Heat Shock Proteins With Peptides And Antigen Presenting Cells Chaperoning of the Innate and Adaptive Immune Responses. Annu Rev Immunol 20:395.
Thurner, B., I. Haendle, C. Roder, D. Dieckmann, P. Keikavoussi, H. Jonuleit, A. Bender, C. Maczek, D. Schreiner, P. von den Driesch, E. B. Brocker, R. M. Steinman, A. Enk, E. Kampgen, and G. Schuler. 1999. Vaccination with mage-3A1 peptide-pulsed mature, monocyte-derived dendritic cells expands specific cytotoxic T cells and induces regression of some metastases in advanced stage 1V melanoma. J Exp Med 190:1669.
Wang, R. F., X. Wang, A. C. Atwood, S. L. Topalian, and S. A. Rosenberg. 1999. Cloning genes encoding MHC class II-restricted antigens: mutated CDC27 as a tumor antigen. Science 284:1351.
Zitvogel, L., A. Regnault, A. Lozier, J. Wolfers, C. Flament, D. Tenza, P. Ricciardi-Castagnoli, G. Raposo, and S. Amigorena. 1998. Eradication of established murine tumors using a novel cell-free vaccine: dendritic cell-derived exosomes. Nat Med 4:594.

1-38. (canceled)

39. A method of treating a cancer patient comprising:
immunizing the patient with a cancer vaccine comprising one or more at least partially mature antigen presenting cells loaded with heat shocked and killed cancer cells that are non-apoptotic.

40. The method of claim 39, wherein the one or more at least partially mature antigen presenting cells are autologous.

41. The method of claim 39, wherein the heat shocked and killed cancer cells are autologous.

42. The method of claim 39, heat shocked and killed cancer cells selected from the cells in Table II.

43. The method of claim 39, wherein the HSP60, HSP90 and gp96 of the cancer cells are upregulated prior to killing.

44. The method of claim 39, wherein the cancer cells are transfected to overexpress HSP60, HSP90 and gp96.

45. The method of claim 39, wherein the cancer cells are killed by betulinic acid, paclitaxel, camptothecin, ellipticine, mithramycin A, etoposide, vinblastine, vincristine, ionomycin and combinations thereof.

46. The method of claim 39, wherein the cancer cells are killed by radiation, heat, cold, osmotic shock, pressure, grinding, shearing, ultrasound, drying, freeze spraying, puncturing, starving and combinations thereof.

47. A method of delivering antigen to dendritic cells in vitro comprising:
contacting dendritic cells capable of internalizing one or more antigens for antigen presentation for a time sufficient to allow the one or more antigens to be internalized for presentation to immune cells, wherein the antigen comprises heat-shocked and killed cancer cells.

48-60. (canceled)
